(19) United States
(12) Patent Application Publication
(10) Pub. No.: US 2021/0252036 A1
(43) Pub. Date: Aug. 19, 2021

(54) METHODS OF TREATING CANCER BY INHIBITING UBIQUITIN CONJUGATING ENZYME E2 K (UBE2K)

(71) Applicant: Berg LLC, Framingham, MA (US)

(72) Inventors: Anne R. Diers, Wilmington, MA (US); Vivek K. Vishnudas, Bedford, MA (US); Stephane Gesta, Concord, MA (US)

(21) Appl. No.: 17/274,405

(22) PCT Filed: Sep. 10, 2019
(86) PCT No.: PCT/US19/50465

§ 371 Date: Mar. 8, 2021

Related U.S. Application Data
(60) Provisional application No. 62/729,348, filed on Sep. 10, 2018.

(51) Int. Cl. A61K 31/713; A61K 31/277; A61K 45/06; A61P 35/00

(52) U.S. Cl. CPC A61K 31/713; A61P 35/00; A61K 45/06; A61K 31/277

(57) ABSTRACT

The disclosure provides methods for the treatment of cancer in a subject comprising administering to the subject an inhibitor of Ubiquitin Conjugating Enzyme E2 K (UBE2K). The UBE2K inhibitor may be administered to the subject as a monotherapy, or in combination with an additional agent, such as an anticancer agent.

### RELATED APPLICATION

This application claims priority to U.S. Provisional Patent Application No. 62/729,348 filed on Sep. 10, 2018, the contents of which are incorporated herein by reference in their entirety.

### SEQUENCE LISTING

The present application contains a Sequence Listing which has been submitted in ASCII format via EFS-Web and is hereby incorporated by reference in its entirety. Said ASCII copy, created on Sep. 9, 2019, is named 119992_19420_Sequence_Listing.txt and is 3 KB in size.

### BACKGROUND

Cancer is presently one of the leading causes of death in developed nations. A diagnosis of cancer traditionally involves serious health complications. Cancer can cause disfigurement, chronic or acute pain, lesions, organ failure, or even death. Traditionally, many cancers are treated with surgery, chemotherapy, radiation, or combinations thereof. Chemotherapeutic agents used in the treatment of cancer are known to produce several serious and unpleasant side effects in patients. For example, some chemotherapeutic agents cause neuropathy, nephrotoxicity (e.g., hyperlipidemia, proteinuria, hypoproteinemia, combinations thereof, or the like), stomatitis, mucositisemesis, alopecia, anorexia, esophagitis amenorrhoea, decreased immunity, anaemia, high tone hearing loss, cardiotoxicity, fatigue, neuropathy, myelosuppression, or combinations thereof. Oftentimes, chemotherapy is not effective, or loses effectiveness after a period of efficacy, either during treatment, or shortly after the treatment regimen concludes (i.e., the treatment regimen does not result in a cure). Improved methods for the treatment of cancer remain desirable.

### SUMMARY OF THE INVENTION

In certain aspects, the disclosure relates to a method of treating cancer in a subject in need thereof, the method comprising administering to the subject a Ubiquitin Conjugating Enzyme E2 K (UBE2K) inhibitor, thereby treating the cancer in the subject.

In certain aspects, the disclosure relates to a method of reducing proliferation of a cancer cell in a subject in need thereof, the method comprising administering to the subject a Ubiquitin Conjugating Enzyme E2 K (UBE2K) inhibitor, thereby reducing proliferation of the cancer cell in the subject relative to a subject that is not administered the UBE2K inhibitor.

In certain aspects, the disclosure relates to a method of inducing death of a cancer cell in a subject in need thereof, the method comprising administering to the subject a Ubiquitin Conjugating Enzyme E2 K (UBE2K) inhibitor, thereby inducing death of the cancer cell in the subject. In certain embodiments, the death of the cancer cell is induced by apoptosis.

In certain embodiments, the UBE2K inhibitor is a specific inhibitor of UBE2K. In certain embodiments, the UBE2K inhibitor comprises a small molecule. In certain embodiments, the UBE2K inhibitor comprises a nucleic acid inhibitor. In certain embodiments, the nucleic acid inhibitor comprises an antisense nucleic acid molecule. In certain embodiments, the nucleic acid inhibitor comprises a double stranded nucleic acid molecule. In certain embodiments, the double stranded nucleic acid molecule comprises a double stranded RNA selected from the group consisting of an siRNA, a shRNA, and a dicer substrate siRNA (DsiRNA). In certain embodiments, the UBE2K inhibitor comprises an antibody.

In certain embodiments, the cancer comprises a solid tumor. In certain embodiments, the solid tumor is selected from the group consisting of carcinoma, melanoma, sarcoma, and lymphoma. In certain embodiments, the solid tumor is selected from the group consisting of pancreatic cancer, liver cancer, colorectal cancer, and lymphoma. In certain embodiments, the solid tumor is pancreatic cancer or liver cancer. In certain embodiments, the cancer is a leukemia.

In certain embodiments, the UBE2K inhibitor is administered with an additional agent. In certain embodiments, the additional agent is an anti-cancer agent. In certain embodiments, the additional agent is a chemotherapeutic agent. In certain embodiments, the chemotherapeutic agent is selected from the group consisting of gemcitabine, 5-fluorouracil, leucovorin, docetaxel, fludarabine, cytarabine, cyclophosphamide, paclitaxel, docetaxel, busulfan, methotrexate, daunorubicin, doxorubicin, melphalan, cladribine, vincristine, vinblastine, chlorambucil, tamoxifen, taxol, camptothecin, actinomycin-D, mitomycin C, combretastatin, cisplatin, etoposide, verapamil, podophyllotoxin, and 5-fluorouracil. In certain embodiments, the additional agent is an anti-angiogenic agent.

In certain embodiments, the additional agent is an immunotherapeutic. In certain embodiments, the immunotherapeutic is an immune checkpoint modulator of an immune checkpoint molecule. In certain embodiments, the immune checkpoint molecule is selected from CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA. In certain embodiments, the immune checkpoint molecule is a stimulatory immune checkpoint molecule and the immune checkpoint modulator is an agonist of the stimulatory immune checkpoint molecule. In certain embodiments, the immune checkpoint molecule is an inhibitory immune checkpoint molecule and the immune checkpoint modulator is an antagonist of the inhibitory immune checkpoint molecule. In certain embodiments, the immune checkpoint modulator is selected from a small molecule, an inhibitory RNA, an antisense molecule, and an immune checkpoint molecule binding protein. In certain embodiments, the immune checkpoint molecule is PD-1 and the immune checkpoint modulator is a PD-1 inhibitor. In certain embodiments, the PD-1 inhibitor is selected from pembrolizumab, nivolumab, pidilizumab, SHR-1210, MEDI0680R01, BBg-A317, TSR-042, REGN2810 and PF-06801591. In certain embodiments, the immune checkpoint molecule is PD-L1 and the immune checkpoint modulator is a PD-L1 inhibitor. In certain embodiments, the PD-L1 inhibitor is selected from durvalumab, atezolizumab, avelumab, MDX-1105, AMP-224 and LY3300054. In certain embodiments, the immune checkpoint molecule is CTLA-4 and the immune checkpoint modulator is a CTLA-4 inhibitor. In certain embodiments, the CTLA-4 inhibitor is selected from ipilimumab, tremelimumab, JMW-3B3 and AGEN1884.

### BRIEF DESCRIPTION OF THE DRAWINGS

FIG. 1 shows an overview of the ubiquitin proteasome system.

FIG. 2 shows UBE2K transcript knock down at 24 h and 96 h post siRNA transfection in Miapaca2, HepG2, and SKHEP1 cells.

FIG. 3 shows UBE2K protein knock down at 24 h and 96 h post siRNA transfection in Miapaca2, HepG2, and SKHEP1 cells.

FIG. 4 shows the effect of UBE2K siRNA mediated knock down on cell number under basal conditions at 96 h post transfection.

FIG. 5 shows the effect of UBE2K siRNA mediated knock down on doxorubicin sensitivity.

FIG. 6 shows the effect of UBE2K siRNA mediated knock down on mRNA expression of related E2s.

FIG. 7 shows the effect of UBE2K siRNA mediated knock down on cell number/viability when cultured in media containing 0.5% or 5% serum.

FIG. 8 shows the effect of UBE2K siRNA mediated knock down on cell death and cell cycle progression.

FIGS. 9A and 9B show regulation of UBE2K interactors at protein level (A) and at transcript level (B) in UBE2K knock down cells versus non targeting siRNA transfected MiaPaca2 cells with and without MG132.

FIG. 10 shows cell cycle analysis of synchronized and unsynchronized populations of MiaPaca2 cells. Pelleted MiaPaca2 cells were treated with Hoechst stain for 10 mins. Cell cycle analysis was analyzed by measuring the MFI of Hoechst staining in FL-3 channel using flow cytometry.

FIG. 11 shows a Cell Titer-Fluor cell viability assay of synchronized MiaPaca2 cells with UBE2K knockdown at 48, 72 and 96 hours after release from serum deprivation with 20% FBS.

FIG. 12 shows changes in the concentrations of cyclins throughout the cell cycle. There is a direct correlation between cyclin accumulation and the three major cell cycle checkpoints.

FIG. 13 shows a time lapse of the levels of key cell cycle regulators in synchronized MiaPaca2 cells.

FIG. 14 shows confirmation of knockdown of Cdc34 and UBE2K in MiaPaca2 cells at 72 hrs post-transfection.

FIG. 15 shows UBE2K knockdown in MiaPaca2 cells led to an approximately 20% reduction in the number of viable cells at 72 hrs post-transfection. Knockdown of Cdc34 had no effect on the number of viable cells.

FIG. 16 shows confirmation of UBE2K knockdown in UBE2K shRNA2 and shRNA3 transduced MiaPaca2 cell lines. Elevated levels of G2/M cell cycle regulatory proteins were observed in UBE2K knockdown cells.

FIG. 17 shows a CellTiter-Fluor cell viability assay after 72 h of plating revealed that the total number of viable cells was significantly lower in the UBE2K knockdown cells than the non-targeting (NT) siRNA control shRNA cells.

FIG. 18 shows direct cell counts and individual nuclei counts in MiaPaca2 cells. UBE2K knockdown cells had a significantly lower number of cells/well at 96 h after plating when compared to the non-targeting (NT) shRNA control counterparts.

FIG. 19 shows a correlation of UBE2K gene expression to survival in pancreatic ductal adenocarcinoma patients.

FIG. 20 shows an example data set for UBE2K immunohistochemical staining in pancreatic tumor tissue vs. normal adjacent tissue.

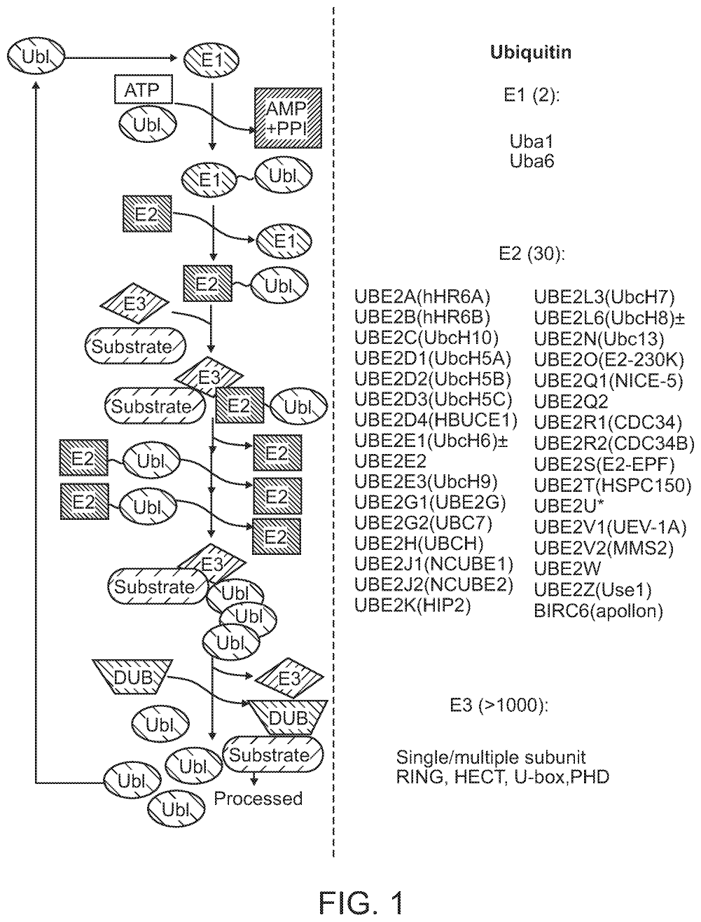

### DETAILED DESCRIPTION AND PREFERRED EMBODIMENTS

A discovery platform technology was used to identify Ubiquitin Conjugating Enzyme E2 K (UBE2K) as a central node that is significantly modulated in human cancer cells. Knockdown of UBE2K in cancer cells, e.g., pancreatic cancer and hepatocellular carcinoma cell lines, reduced cancer cell numbers in vitro, and further studies indicated that UBE2K knockdown reduced cancer cell proliferation and induced cancer cell death. Accordingly, the present invention provides methods of reducing proliferation and/or inducing death of a cancer cell by administering a UBE2K inhibitor. The present invention further provides methods of treating a cancer in a subject by administering to the subject a UBE2K inhibitor.

### I. Definitions

The terms “administer”, “administering” or “administration” include any method of delivery of a pharmaceutical composition or agent into a subject's system or to a particular region in or on a subject. In certain embodiments, the agent is delivered orally. In certain embodiments, the agent is administered parenterally. In certain embodiments, the agent is delivered topically including transmucosally. In certain embodiments, the agent is delivered by inhalation. In certain embodiments of the invention, an agent is administered by parenteral delivery, including, intravenous, intramuscular, subcutaneous, intramedullary injections, as well as intrathecal, direct intraventricular, intraperitoneal, intranasal, or intraocular injections. In one embodiment, the compositions provided herein may be administered by injecting directly to a tumor. In some embodiments, the agent is delivered by injection or infusion. In certain embodiments, administration is systemic. In certain embodiments, administration is local. In some embodiments, one or more routes of administration may be combined, such as, for example, intravenous and intratumoral, or intravenous and peroral, or intravenous and oral, intravenous and topical, or intravenous and transdermal or transmucosal. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (e.g., Fab, Fab′, F(ab′)₂, and Fv fragments), single chain Fv (scFv) mutants, multispecific antibodies such as bispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc

In some embodiments, an antibody is a non-naturally occurring antibody. In some embodiments, an antibody is purified from natural components. In some embodiments, an antibody is recombinantly produced. In some embodiments, an antibody is produced by a hybridoma.

The term “antibody fragment” refers to a portion of an intact antibody and refers to the antigenic determining variable regions of an intact antibody. Examples of antibody fragments include, but are not limited to, Fab, Fab′, F(ab′)₂, and Fᵥ fragments, linear antibodies, single chain antibodies, and multispecific antibodies formed from antibody fragments. The term “antigen-binding fragment” of an antibody includes one or more fragments of an antibody that retain the ability to specifically bind to an antigen. It has been shown that the antigen-binding function of an antibody can be performed by certain fragments of a full-length antibody. Examples of binding fragments encompassed within the term “antigen-binding fragment” of an antibody include (without limitation): (i) an Fab fragment, a monovalent fragment consisting of the V_{L}, V_{H}, C_{L}, and C_{H1} domains (e.g., an antibody digested by papain yields three fragments: two antigen-binding Fab fragments, and one Fc fragment that does not bind antigen); (ii) a F(ab′)₂ fragment, a bivalent fragment comprising two Fab fragments linked by a disulfide bridge at the hinge region (e.g., an antibody digested by pepsin yields two fragments: a bivalent antigen-binding F(ab′)₂ fragment, and a pFc′ fragment that does not bind antigen) and its related F(ab′) monovalent unit; (iii) a F_{d} fragment consisting of the V_{H} and C_{H1} domains (i.e., that portion of the heavy chain which is included in the Fab); (iv) a Fᵥ fragment consisting of the V_{L} and V_{H} domains of a single arm of an antibody, and the related disulfide linked Fᵥ; (v) a dAb (domain antibody) or sdAb (single domain antibody) fragment (Ward et al., Nature 341:544-546, 1989), which consists of a V_{H} domain; and (vi) an isolated complementarity determining region (CDR).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers found that certain sub-portions within Kabat CDRs adopt nearly identical peptide backbone conformations, despite having great diversity at the level of amino acid sequence (Chothia et al. (1987) J. MOL. BIOL. 196: 901-917, and Chothia et al. (1989) NATURE 342: 877-883). These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139, and MacCallum et al. (1996) J. MOL. BIOL. 262 (5): 732-45. Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, a “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)₂, Fᵥ), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody”, as used herein refers to non-human (e.g., murine) antibodies that are chimeric immunoglobulins, immunoglobulin chains, or fragments thereof (such as Fv, Fab, Fab′, F(ab′)2 or other antigen-binding subsequences of antibodies) which contain minimal sequence derived from a non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al. (1986)

NATURE 321: 522-525; Reichmann et al. (1988) NATURE 332: 323-329; and Presta (1992) CURR. OP. STRUCT. BIOL. 2: 593-596, each of which is incorporated by reference herein in its entirety.

A “bivalent antibody” refers to an antibody or antigen-binding fragment thereof that comprises two antigen-binding sites. The two antigen binding sites may bind to the same antigen, or they may each bind to a different antigen, in which case the antibody or antigen-binding fragment is characterized as “bispecific.” A “tetravalent antibody” refers to an antibody or antigen-binding fragment thereof that comprises four antigen-binding sites. In certain embodiments, the tetravalent antibody is bispecific. In certain embodiments, the tetravalent antibody is multispecific, i.e. binding to more than two different antigens.

Fab (fragment antigen binding) antibody fragments are immunoreactive polypeptides comprising monovalent antigen-binding domains of an antibody composed of a polypeptide consisting of a heavy chain variable region (V_{H}) and heavy chain constant region 1 (C_{H1}) portion and a poly peptide consisting of a light chain variable (V_{L}) and light chain constant (C_{L}) portion, in which the C_{L} and C_{H1} portions are bound together, preferably by a disulfide bond between Cys residues.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features.

As used herein, “cancer” refers to all types of cancer or neoplasm or malignant tumors found in humans, including, but not limited to: leukemias, lymphomas, melanomas, carcinomas and sarcomas. As used herein, the terms or language “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells. In certain embodiments, the cancer is a solid tumor. In certain embodiments, the cancer is a blood tumor (i.e., a non-solid tumor).

A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

Specific criteria for the staging of cancer are dependent on the specific cancer type based on tumor size, histological characteristics, tumor markers, and other criteria known by those of skill in the art. Generally, cancer stages can be described as follows:

### Stage 0—Carcinoma in situ

Stage I, Stage II, and Stage III—Higher numbers indicate more extensive disease: Larger tumor size and/or spread of the cancer beyond the organ in which it first developed to nearby lymph nodes and/or tissues or organs adjacent to the location of the primary tumor

### Stage IV—The Cancer Has Spread to Distant Tissues or Organs

RECIST criteria are clinically accepted assessment criteria used to provide a standard approach to solid tumor measurement and provide definitions for objective assessment of change in tumor size for use in clinical trials. Such criteria can also be used to monitor response of an individual undergoing treatment for a solid tumor. The RECIST 1.1 criteria are discussed in detail in Eisenhauer et al. (New response evaluation criteria in solid tumors: Revised RECIST guideline (version 1.1) Eur. J. Cancer. 45:228-247, 2009), the entire contents of which are incorporated herein by reference. Response criteria for target lesions include:

Complete Response (CR): Disappearance of all target lesions. Any pathological lymph nodes (whether target or non-target) must have a reduction in short axis to <10 mm.

Partial Response (PR): At least a 30% decrease in the sum of diameters of target lesion, taking as a reference the baseline sum diameters.

Progressive Diseases (PD): At least a 20% increase in the sum of diameters of target lesions, taking as a reference the smallest sum on the study (this includes the baseline sum if that is the smallest on the study). In addition to the relative increase of 20%, the sum must also demonstrate an absolute increase of at least 5 mm. (Note: the appearance of one or more new lesions is also considered progression.)

Stable Disease (SD): Neither sufficient shrinkage to qualify for PR nor sufficient increase to qualify for PD, taking as a reference the smallest sum diameters while on study.

RECIST 1.1 criteria also consider non-target lesions which are defined as lesions that may be measureable, but need not be measured, and should only be assessed qualitatively at the desired time points. Response criteria for non-target lesions include:

Complete Response (CR): Disappearance of all non-target lesions and normalization of tumor marker levels. All lymph nodes must be non-pathological in size (<10 mm short axis).

Non-CR/Non-PD: Persistence of one or more non-target lesion(s) and/or maintenance of tumor marker level above the normal limits.

Progressive Disease (PD): Unequivocal progression (emphasis in original) of existing non-target lesions. The appearance of one or more new lesions is also considered progression. To achieve “unequivocal progression” on the basis of non-target disease, there must be an overall level of substantial worsening of non-target disease such that, even in the presence of SD or PR in target disease, the overall tumor burden has increased sufficiently to merit discontinuation of therapy. A modest “increase” in the size of one or more non-target lesions is usually not sufficient to qualify for unequivocal progression status. The designation of overall progression solely on the basis of change in non-target disease in the face of SD or PR in target disease will therefore be extremely rare.

Clinically acceptable criteria for response to treatment in acute leukemias are as follows:

Complete remission (CR): The patient must be free of all symptoms related to leukemia and have an absolute neutrophil count of ≥1.0×10⁹/L, platelet count ≥100×10⁹/L, and normal bone marrow with <5% blasts and no Auer rods.

Complete remission with incomplete blood count recovery (Cri): As per CE, but with residual thrombocytopenia (platelet count <100×10⁹/L) or residual neutropenia (absolute neutrophil count <1.0×10⁹/L).

Partial remission (PR): A ≥50% decrease in bone marrow blasts to 5 to 25% abnormal cells in the marrow; or CR with ≤5% blasts if Auer rods are present.

Treatment failure: Treatment has failed to achieve CR, Cri, or PR. Recurrence.

Relapse after confirmed CR: Reappearance of leukemic blasts in peripheral blood or ≥5% blasts in the bone marrow not attributable to any other cause (e.g., bone marrow regeneration after consolidated therapy) or appearance of new dysplastic changes.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

A “chemotherapeutic regimen” is a clinically accepted dosing protocol for the treatment of cancer that includes administration of one or more chemotherapeutic agents to a subject in specific amounts on a specific schedule. In certain embodiments, the chemotherapeutic agent can be an agent in clinical trials.

Chemotherapeutic regimens can include administration of a drug on a predetermined “cycle” including intervals of dosing and not dosing with one or more agents for the treatment of cancer. For example, an agent can be administered one or more times per week for three consecutive weeks followed by a week of no agent administered to provide a four week cycle. The cycle can be repeated so that the subject would be subjected to three treatment weeks, one no treatment week, three treatment weeks, one no treatment week, etc., for the desired number of cycles. In certain embodiments, treatment of efficacy and laboratory values (e.g., liver enzymes, blood count, kidney function) are assessed at the end of each cycle or every other cycle.

An “immunotherapeutic” as used herein refers to a pharmaceutically acceptable compound, composition or therapy that induces or enhances an immune response. Immunotherapeutics include, but are not limited to, immune checkpoint modulators, Toll-like receptor (TLR) agonists, cell-based therapies, cytokines and cancer vaccines.

As used herein, an “immune checkpoint” or “immune checkpoint molecule” is a molecule in the immune system that modulates a signal. An immune checkpoint molecule can be a stimulatory checkpoint molecule, i.e., increase a signal, or inhibitory checkpoint molecule, i.e., decrease a signal. A “stimulatory checkpoint molecule” as used herein is a molecule in the immune system that increases a signal or is co-stimulatory. An “inhibitory checkpoint molecule”, as used herein is a molecule in the immune system that decreases a signal or is co-inhibitory.

As used herein, an “immune checkpoint modulator” is an agent capable of altering the activity of an immune checkpoint in a subject. In certain embodiments, an immune checkpoint modulator alters the function of one or more immune checkpoint molecules including, but not limited to, CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA. The immune checkpoint modulator may be an agonist or an antagonist of the immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In other embodiments, the immune checkpoint modulator is a small molecule. In a particular embodiment, the immune checkpoint modulator is an anti-PD1, anti-PD-L1, or anti-CTLA-4 binding protein, e.g., antibody or antibody fragment.

As used herein, the terms “increasing” (or “activating”) and “decreasing” refer to modulating resulting in, respectively, greater or lesser amounts, function or activity of a parameter relative to a reference. For example, subsequent to administration of a UBE2K inhibitor (e.g. a specific inhibitor of UBE2K) described herein, a parameter (e.g. tumor size, cancer cell proliferation, or cancer cell death) may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration, or relative to a subject or cancer cell that is not administered the UBE2K inhibitor. Generally, the metric is measured subsequent to administration at a time that the administration has had the recited effect, e.g., at least one day, one week, one month, 3 months, 6 months, after a treatment regimen has begun. In addition, the metric may be measured relative to a cancer cell or subject that is not administered the UBE2K inhibitor (e.g. a specific inhibitor of UBE2K). Similarly, pre-clinical parameters (such as death or proliferation of cancer cells in vitro, and/or reduction in UBE2K activity, by a UBE2K inhibitor described herein) may be increased or decreased by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the parameter prior to administration of the UBE2K inhibitor (e.g. a specific inhibitor of UBE2K), or relative to a cancer cell or UBE2K enzyme that is not treated with the UBE2K inhibitor (e.g. a specific inhibitor of UBE2K).

As used herein, a decrease in the expression or activity of UBE2K is understood to include a change in expression or activity of the gene and/or the protein. In an embodiment, expression or activity is reduced by at least about 10%, 20%, 30%, 40%, 50%, 60%, 70%, 75%, 80%, 85%, 90%, 95%, or 99%.

As used herein, a “nucleic acid” UBE2K inhibitor is any nucleic acid-based inhibitor that causes a decrease in the expression and/or activity of UBE2K. In certain embodiments, a nucleic acid inhibitor acts by hybridizing with at least a portion of the RNA transcript from the corresponding gene (UBE2K) to result in a decrease in the expression of UBE2K. Nucleic acid inhibitors include, for example, single stranded nucleic acid molecules, e.g., antisense nucleic acids, and double stranded nucleic acids such as siRNA, shRNA, dsiRNA (see, e.g., US Patent Publication No. US2007/0104688). As used herein, double stranded nucleic acid molecules are designed to be double stranded over at least 12, preferably at least 15 nucleotides. Double stranded nucleic acid molecules can be a single nucleic acid strand designed to hybridize to itself, e.g., an shRNA. It is understood that a nucleic acid inhibitor can be administered as an isolated nucleic acid. Alternatively, the nucleic acid inhibitor can be administered as an expression construct to produce the inhibitor in the cell. In certain embodiments, the nucleic acid inhibitor includes one or more chemical modifications to improve the activity and/or stability of the nucleic acid inhibitor. Such modifications are well known in the art. The specific modifications to be used will depend, for example, on the type of nucleic acid inhibitor. In certain embodiments, the nucleic acid UBE2K inhibitor is a specific inhibitor of UBE2K, i.e. does not decrease expression or activity of other genes or proteins besides UBE2K.

As used herein, a “small molecule” UBE2K inhibitor is a UBE2K inhibitor molecule that has a molecular weight of less than 1000 Da, preferably less than 750 Da, or preferably less than 500 Da. In certain embodiments, a “small molecule” is a synthetic organic compound and does not include a nucleic acid molecule. In certain embodiments, a “small molecule” is a synthetic organic compound and does not include a peptide more than three amino acids in length. A “small molecule” UBE2K inhibitor is a molecule that specifically binds to UBE2K and at least partially inhibits UBE2K. In certain embodiments, the small molecule UBE2K inhibitor is a specific inhibitor of UBE2K, i.e. does not substantially decrease expression or activity of other genes or proteins besides UBE2K. For example, in some embodiments, the small molecule UBE2K inhibitor does not reduce activity of nuclear factor kappa-light-chain-enhancer of activated B cells (NF kappa B).

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

A “therapeutically effective amount” is that amount sufficient to treat a disease in a subject. A therapeutically effective amount can be administered in one or more administrations.

As used herein, the terms “treat,” “treating” or “treatment” refer, preferably, to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the quality of life (QOL) as judged by a qualified individual, e.g., a treating physician, e.g., using accepted assessment tools of pain and QOL is not considered to be a “treatment” of the cancer.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein including, but not limited to, combinations of dosing rates, dosing times, dosing amounts, treatment methods, monitoring methods, and selection methods.

### II. UBE2K

UBE2K is a component of ubiquitin dependent proteolysis, which is responsible for protein degradation. Ubiquitin dependent proteolysis utilizes ubiquitin-activating (E1), ubiquitin-conjugating (E2), and ubiquitin-ligating (E3) enzymes that sequentially activate, transfer and ligate ubiquitin (Ub) onto a lysine residue of a target protein. During the middle step of this cascade, ubiquitin forms a covalent thioester complex (E2-Ub) between its C-terminal carboxylate and a catalytic cysteine in the E2 enzyme. Transfer to a substrate is then mediated by either a RING E3 ligase that acts as a scaffolding protein or a HECT E3 ligase that forms a further covalent thioester complex with ubiquitin prior to substrate labeling. Repeated cycles of this process form a polyubiquitin chain using one or more of the seven available lysine residues found on ubiquitin (ie. K6, K11, K27, K29, K33, K48 and/or K63). See Cook et al., 2015, PLoS ONE 10 (3): e0120318.

UBE2K is a protein component of this ubiquitin proteasome system and is well characterized as an enzyme responsible for ubiquitination of various proteins. It is also known as Huntington interacting protein 2 (HIP2). UBE2K, one of the approximately 50 E2 enzymes of the ubiquitin system, is a 25 kDa protein. The human UBE2K amino acid and nucleic acid sequences are provided herein as SEQ ID NO: 1 and SEQ ID NO: 2, respectively. UBE2K functions to accept ubiquitin moieties from El enzyme and covalently link them to other protein substrates via a thioester bond, marking them for degradation by the proteasome. It is also known to interact with ubiquitin non-covalently. It is known to synthesize Lys 48 linked polyubiquitin chains and is a member of the Class III E2 family. It is unique among other E2s, as in addition to the conserved UBC domain, it has a C terminal ubiquitin associated (UBA) domain. UBA domain of UBE2K has been reported to direct the specificity in polyubiquitin chain linkage. It also increases the processivity in Lys 48 linked polyubiquitin chain synthesis. Using a yeast two-hybrid screen, UBE2K was found to interact with several ring finger proteins. It was also found that ring finger domain of RNF2 is essential for the recognition of UBE2K, and RNF2 plays a role as an E3 ligase. In addition, UBE2K has the ability to ubiquitinate substrates independently of E3 ligase. In vitro ubiquitination assays indicated that mutation of active site cysteine residue in UBC domain impairs the ability of UBE2K to make mono or polyubiquitin chains.

Although there is extensive literature on the ubiquitin proteasome system in cancer, there are fewer reports on the role of specific E2 enzymes. Importantly, UBE2K is reported to work in concert with two relevant tumor suppressors to regulate cancer-relevant phenotypes. UBE2K interacts with BRCA1 and ubiquitinates G2/M cell cycle proteins cyclin B land cdc25, affecting proliferation, while BRCA1 acts as an E3 ligase in this setting. In addition, UBE2K has been shown to ubiquitinate p53 by using MDM2 as an E3 ligase and promote its degradation.

UBE2K activity may be assayed by incubating the UBE2K enzyme with fluorescently labeled ubiquitin followed by SDS-PAGE of the ubiquitinated complex. For example, UBE2K may be incubated in buffer containing fluorescently labeled ubiquitin, followed by addition of magnesium acetate and ATP. The reaction may be terminated by the addition of 2.5 μl of 10% (w/v) SDS and heating for 6 min at 75° C. The samples are then subjected to SDS-PAGE in the absence of any thiol. Methods for assaying UBE2K activity are described, for example, by Cook et al., 2015, PLoS ONE 10 (3): e0120318; Middleton et al., Sci. Rep. (5):16793; and Strickson et al., 2013, Biochem Journal 451: 427-437 each of which is incorporated by reference herein in its entirety.

### III. UBE2K Inhibitors

As used herein, a UBE2K inhibitor refers to a small molecule compound (i.e., a synthetic organic compound), a nucleic acid (e.g., antisense, siRNA, shRNA, dsiRNA, etc.), a protein, or an antibody (e.g. a protein or antibody that specifically binds to the enzyme UBE2K) that partially or fully inhibits the enzyme by reducing the expression and/or activity of the enzyme, for example, by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 50-fold, at least 75-fold, or at least 100-fold, etc; or the UBE2K expression and/or activity is reduced by at least about 10%, 20%, 30%, 40%, 50%, 60%, 70%, 75%, 80%, 85%, 90%, 95%, or 99%. As used herein, a “UBE2K inhibitor” can act by any mechanism, e.g., by inhibiting the expression of UBE2K at the RNA or protein level; or by inhibiting the activity of UBE2K, e.g., by inhibiting the loading of ubiquitin onto UBE2K. In preferred embodiments, the UBE2K inhibitor is a specific inhibitor of UBE2K, i.e. does not substantially reduce the expression or activity of polypeptides other than UBE2K. In some embodiments, the UBE2K specific inhibitor preferentially inhibits UBE2K as compared to one or more different E2 proteins (not UBE2K) by at least 5-fold, at least 10-fold, at least 20-fold, at least 30-fold, at least 50-fold, at least 75-fold, at least 100-fold, at least 500-fold, or at least 1000-fold. In some embodiments, the UBE2K that is inhibited is human UBE2K. In some embodiments, the UBE2K inhibitor does not reduce activity of nuclear factor kappa-light-chain-enhancer of activated B cells (NF kappa B). In some embodiments, the UBE2K inhibitor does not reduce the expression or activity of other E2 enzymes besides UBE2K.

A UBE2K inhibitor may be identified using methods known in the art. For example, compounds that reduce UBE2K expression may be identified by treating cells in vitro with a putative UBE2K inhibitor and measuring its effect on UBE2K expression, e.g. mRNA or protein expression. Protein levels of UBE2K may be measured by suitable techniques known in the art including ELISA or Western blot. The level of a UBE2K nucleic acid (e.g. an mRNA) may be measured using suitable techniques known in the art including polymerase chain reaction (PCR) amplification reaction, reverse-transcriptase PCR analysis, quantitative real-time PCR, single-strand conformation polymorphism analysis (SSCP), mismatch cleavage detection, heteroduplex analysis, Northern blot analysis, in situ hybridization, array analysis, deoxyribonucleic acid sequencing, restriction fragment length polymorphism analysis, and combinations or sub-combinations thereof.

UBE2K inhibitors may also be identified by measuring their effect on UBE2K activity using methods known in the art such as those described above. For example, a putative UBE2K inhibitor may be incubated with UBE2K enzyme and fluorescently labeled ubiquitin to determine whether the putative inhibitor prevents ubiquitination of the UBE2K enzyme. Following incubation, the samples are subject to SDS-PAGE and ubiquitin/UBE2K complexes are detected.

### A. Small Molecule UBE2K Inhibitors

Small molecule UBE2K inhibitors include (E)3-[(4-Methylphenyl)sulfonyl]-2-propenenitrile (BAY 11-7082). This compound has been shown to inhibit the loading of ubiquitin onto E2 conjugating enzymes such as UBE2K. See Strickson et al., 2013, Biochem Journal 451: 427-437, which is incorporated by reference herein in its entirety. BAY11-7082 is described, for example, in US 2005/0124590 and US 2009/0082371, each of which is incorporated by reference herein in its entirety. The chemical structure of BAY 11-7082 is provided below.

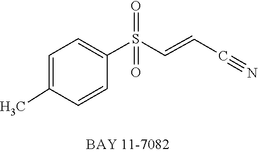

In some embodiments, the UBE2K inhibitor does not comprise BAY 11-7082. In some embodiments, the UBE2K inhibitor does not comprise a small molecule.

### B. Nucleic Acid UBE2K Inhibitors

In some embodiments, the UBE2K inhibitor (e.g. a specific inhibitor of UBE2K) is a nucleic acid. Nucleic acid inhibitors are well known in the art. Nucleic acid inhibitors include both single stranded and double stranded nucleic acids (i.e., nucleic acids having a complementary region of at least 15 nucleotides in length) that are complementary to a target sequence in a cell. Nucleic acids can be delivered to a cell in culture, e.g., by adding the nucleic acid to culture media either alone or with an agent to promote uptake of the nucleic acid into the cell. Nucleic acids can be delivered to a cell in a subject, i.e., in vivo, by any route of administration. The specific formulation will depend on the route of administration.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

Sequences can be “fully complementary” with respect to each when there is base-pairing of the nucleotides of the first nucleotide sequence with the nucleotides of the second nucleotide sequence over the entire length of the first and second nucleotide sequences. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs as is common in double stranded nucleic acid therapeutics, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes described herein.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The terms “complementary,” “fully complementary” and “substantially complementary” herein may be used with respect to the base matching between the sense strand and the antisense strand of a dsRNA, or between an antisense nucleic acid or the antisense strand of dsRNA and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding HSP90, especially HSP90β) including a 5′ UTR, an open reading frame (ORF), or a 3′ UTR. For example, a polynucleotide is complementary to at least a part of UBE2K mRNA if the sequence is substantially complementary to a non-interrupted portion of an mRNA encoding UBE2K.

Patents directed to antisense nucleic acids, chemical modifications, and therapeutic uses are provided, for example, in U.S. Pat. No. 5,898,031 related to chemically modified RNA-containing therapeutic compounds, and U.S. Pat. No. 6,107,094 related methods of using these compounds as therapeutic agent. U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality RNA nucleosides and at least one chemical modification. Each of the patents listed in the paragraph are incorporated herein by reference.

Nucleic acid inhibitors may include natural (i.e. A, G, U, C, or T) or modified (7-deazaguanosine, inosine, etc.) bases. In addition, the bases in nucleotide may be joined by a linkage other than a phosphodiester bond, so long as it does not interfere with hybridization. Thus, inhibitory nucleic acids may be peptide nucleic acids in which the constituent bases are joined by peptide bonds rather than phosphodiester linkages. The inhibitory nucleic acids may be prepared by converting the RNA to cDNA using known methods (see, e.g., Ausubel et. al., Current Protocols in Molecular Biology Wiley 1999). The inhibitory nucleic acids can also be cRNA (see, e.g., Park et. al., (2004) Biochem. Biophys. Res. Commun. 325 (4):1346-52).

Nucleic acid inhibitors can be produced from synthetic methods such as phosphoramidite methods, H-phosphonate methodology, and phosphite trimester methods. Inhibitory nucleic acids can also be produced by PCR methods. Such methods produce cDNA and cRNA sequences complementary to the mRNA. The method of synthesis of a therapeutic nucleic acid is not a limitation of the invention.

Nucleic acid inhbitors typically include one or more chemical modifications to improve their stability and to modulate their pharmacokinetic and pharmacodynamic properties. For example, the modifications on the nucleotides can include, but are not limited to, LNA, HNA, CeNA, 2′-methoxyethyl, 2′-O-alkyl, 2′-O-allyl, 2′-C-allyl, 2′-fluoro, 2′-deoxy, 2′-hydroxyl, and combinations thereof.

Nucleic acid inhibitors may further comprise at least one phosphorothioate or methylphosphonate internucleotide linkage. The phosphorothioate or methylphosphonate internucleotide linkage modification may occur on any nucleotide of the sense strand or antisense strand or both (in nucleic acid therapeutics including a sense strand) in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

Other modifications include the incorporation of modified bases (or modified nucleoside or modified nucleotides) that are variations of standard bases, sugars and/or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). The aptamers may also include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. The aptamer may further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanine, 7-methylguanine, 8-azaguanine, 7-deazaguanine or 3-deazaguanine. The aptamer may still further include adenine or any adenine-related base including 6-methyladenine, N6-isopentenyladenine, N6-methyladenine, 1-methyladenine, 2-methyladenine, 2-methylthio-N6-isopentenyladenine, 8-haloadenine (e.g., 8-fluoroadenine, 8-bromoadenine, 8-chloroadenine, and 8-iodoadenine), 8-aminoadenine, 8-sulfhydryladenine, 8-thioalkyladenine, 8-hydroxyladenine, 7-methyladenine, 2-haloadenine (e.g., 2-fluoroadenine, 2-bromoadenine, 2-chloroadenine, and 2-iodoadenine), 2-amino adenine, 8-azaadenine, 7-deazaadenine or 3-deazaadenine. Also included are uracil or any uracil-related base including 5-halouracil (e.g., 5-fluorouracil, 5-bromouracil, 5-chlorouracil, 5-iodouracil), 5-(carboxyhydroxylmethyl)uracil, 5-carboxymethylaminomethyl-2-thiouracil, 5-carboxymethylaminomethyluracil, dihydrouracil, 1-methylpseudouracil, 5-methoxyaminomethyl-2-thiouracil, 5′-methoxycarbonylmethyluracil, 5-methoxyuracil, 5-methyl-2-thiouracil, 2-thiouracil, 4-thiouracil, 5-methyluracil, uracil-5-oxyacetic acid methylester, uracil-5-oxyacetic acid, pseudouracil, 5-methyl-2-thiouracil, 2-thiouracil, 3-(3-amino-3-N-2-carboxypropyl)uracil, 5-methylaminomethyluracil, 5-propynyl uracil, 6-azouracil, or 4-thiouracil.

Examples of other modified base variants known in the art include, without limitation, e.g., 4-acetylcytidine, 5-(carboxyhydroxylmethyl) uridine, 2′-methoxycytidine, 5-carboxymethylaminomethyl-2-thioridine, 5-carboxymethylaminomethyluridine, dihydrouridine, 2′-O-methylpseudouridine, b-D-galacto sylqueosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, b-D-mannosylqueosine, 5-methoxycarbonylmethyluridine, 5-methoxyuridine, 2-methylthio-N6-isopentenyladenosine, N-((9-b-D-ribofuranosyl-2-methylthiopurine-6-yl)carbamoyl)threonine, N-((9-b-D-ribofuranosylpurine-6-yl)N-methyl-carbamoyl)threonine, urdine-5-oxyacetic acid methylester, uridine-5-oxyacetic acid (v), wybutoxosine, pseudouridine, queosine, 2-thiocytidine, 5-methyl-2-thiouridine, 2-thiouridine, 4-thiouridine, 5-methyluridine, N-((9-b-D-ribofuranosylpurine-6-yl)carbamoyl)threonine, 2′-O-methyl-5-methyluridine, 2′-O-methyluridine, and wybutosine, 3-(3-amino-3-carboxypropyl)uridine.

Also included are the modified nucleobases described in U.S. Pat. Nos. 3,687,808, 3,687,808, 4,845,205, 5,130,302, 5,134,066, 5,175,273, 5,367,066, 5,432,272, 5,457,187, 5,459,255, 5,484,908, 5,502,177, 5,525,711, 5,552,540, 5,587,469, 5,594,121, 5,596,091, 5,614,617, 5,645,985, 5,830,653, 5,763,588, 6,005,096, and 5,681,941, each of which is incorporated herein by reference in its entirety. Examples of modified nucleoside and nucleotide sugar backbone variants known in the art include, without limitation, those having, e.g., 2′ ribosyl substituents such as F, SH, SCH₃, OCN, Cl, Br, CN, CF₃, OCF₃, SOCH₃, SO₂, CH₃, ONO₂, NO₂, N₃, NH₂, OCH₂CH₂OCH₃, O(CH₂)₂ON(CH₃)₂, OCH₂OCH₂N(CH₃)₂, O(C₁₋₁₀ alkyl), O(C₂₋₁₀ alkenyl), O(C₂₋₁₀ alkynyl), S(C₁₋₁₀ alkyl), S(C₂₋₁₀ alkenyl), S(C2-10 alkynyl), NH(C1-10 alkyl), NH(C₂₋₁₀ alkenyl), NH(C2-10 alkynyl), and O-alkyl-O-alkyl. Desirable 2′ ribosyl substituents include 2′-methoxy (2′-OCH₃), 2′-aminopropoxy (2′ OCH₂CH₂CH₂NH₂), 2′-O-allyl(2′-CH₂—CH═CH₂), 2′-O-allyl(2′-O—CH₂—CH═CH₂), 2′-amino (2′-NH₂), and 2′-fluoro (2′-F). The 2′-substituent may be in the arabino (up) position or ribo (down) position.

### Single Stranded Nucleic Acid Therapeutics

Antisense nucleic acid therapeutic agents are single stranded nucleic acid therapeutics, typically about 16 to 30 nucleotides in length, and are complementary to a target nucleic acid sequence in the target cell, either in culture or in an organism.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. For example, the antisense RNA molecule may have a sequence of at least 15, 16, 17, 18, 19, 20 or more contiguous nucleotides that are complementary to the target mRNA.

Patents directed to antisense nucleic acids, chemical modifications, and therapeutic uses are provided, for example, in U.S. Pat. No. 5,898,031 related to chemically modified RNA-containing therapeutic compounds, and U.S. Pat. No. 6,107,094 related methods of using these compounds as therapeutic agent. U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality RNA nucleosides and at least one chemical modification. The entire contents of each of the patents listed in this paragraph are incorporated herein by reference.

### Double Stranded Nucleic Acid Therapeutics

Nucleic acid inhibitors also include double stranded nucleic acids. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi agent may comprise one or more nucleotide overhangs. The term “siRNA” is also used herein to refer to an RNAi agent as described above.

In many embodiments, the duplex region is 15-30 nucleotide pairs in length. In some embodiments, the duplex region is 17-23 nucleotide pairs in length, 17-25 nucleotide pairs in length, 23-27 nucleotide pairs in length, 19-21 nucleotide pairs in length, or 21-23 nucleotide pairs in length.

In certain embodiments, each strand has 15-30 nucleotides.

The RNAi agents that are used in the methods of the invention include agents with chemical modifications as disclosed, for example, in U.S. Provisional Application No. 61/561,710, filed on Nov. 18, 2011, International Application No. PCT/US2011/051597, filed on Sep. 15, 2010, and PCT Publication WO 2009/073809, the entire contents of each of which are incorporated herein by reference. The term “antisense strand” refers to the strand of a double stranded RNAi agent which includes a region that is substantially complementary to a target sequence (e.g., a human TTR mRNA). As used herein, the term “region complementary to part of an mRNA encoding transthyretin” refers to a region on the antisense strand that is substantially complementary to part of a TTR mRNA sequence. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

### C. Antibody UBE2K Inhibitors

In some embodiments, the inhibitor that reduces the expression or activity of UBE2K is an antibody. Therapeutic methods of the invention can include the use of antibodies, including polyclonal and monoclonal antibodies. The term “monoclonal antibody” or “monoclonal antibody composition”, as used herein, refers to a population of antibody molecules that contain only one species of an antigen binding site capable of immunoreacting with a particular epitope. In some embodiments, the antibodies for use in the methods described herein specifically bind to UBE2K, i.e. do not bind to other polypeptides besides UBE2K. Antibodies can be obtained from commercial sources or produced using known methods.

Polyclonal antibodies can be prepared by immunizing a suitable subject with a protein of the invention as an immunogen. The antibody titer in the immunized subject can be monitored over time by standard techniques, such as with an enzyme linked immunosorbent assay (ELISA) using immobilized polypeptide. At an appropriate time after immunization, e.g., when the specific antibody titers are highest, antibody-producing cells can be obtained from the subject and used to prepare monoclonal antibodies (mAb) by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternatively to preparing monoclonal antibody-secreting hybridomas, a monoclonal antibody directed against a protein of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Recombinant antibodies that specifically bind a protein of interest (i.e. UBE2K) can also be used in the methods of the invention. In preferred embodiments, the recombinant antibodies specifically bind a protein of interest (i.e. UBE2K) or fragment thereof. Recombinant antibodies include, but are not limited to, chimeric and humanized monoclonal antibodies, comprising both human and non-human portions, single-chain antibodies and multi-specific antibodies. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Single-chain antibodies have an antigen binding site and consist of a single polypeptide. They can be produced by techniques known in the art, for example using methods described in Ladner et. al U.S. Pat. No. 4,946,778 (which is incorporated herein by reference in its entirety); Bird et al., (1988) Science 242:423-426; Whitlow et al., (1991) Methods in Enzymology 2:1-9; Whitlow et al., (1991) Methods in Enzymology 2:97-105; and Huston et al., (1991) Methods in Enzymology Molecular Design and Modeling: Concepts and Applications 203:46-88. Multi-specific antibodies are antibody molecules having at least two antigen-binding sites that specifically bind different antigens. Such molecules can be produced by techniques known in the art, for example using methods described in Segal, U.S. Pat. No. 4,676,980 (the disclosure of which is incorporated herein by reference in its entirety); Holliger et al., (1993) Proc. Natl. Acad. Sci. USA 90:6444-6448; Whitlow et al., (1994) Protein Eng. 7:1017-1026 and U.S. Pat. No. 6,121,424.

Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Patent No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

More particularly, humanized antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

The antibodies of the invention can be isolated after production (e.g., from the blood or serum of the subject) or synthesis and further purified by well-known techniques. For example, IgG antibodies can be purified using protein A chromatography. Antibodies specific for a protein of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein of the invention.

An antibody directed against a protein (i.e. UBE2K) can be used to isolate the protein by standard techniques, such as affinity chromatography or immunoprecipitation. Moreover, such an antibody can be used to detect the target protein, e.g., UBE2K, or fragment thereof (e.g., in a cellular lysate or cell supernatant) in order to evaluate the level and pattern of expression of the target protein. The antibodies can also be used diagnostically to monitor protein levels in tissues or body fluids (e.g. in disease sate or toxicity state associated body fluid) as part of a clinical testing procedure, e.g., to, for example, determine the efficacy of a given treatment regimen. Detection can be facilitated by the use of an antibody derivative, which comprises an antibody of the invention coupled to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### IV. Treatment of Cancer

In some embodiments, the disclosure provides methods for the treatment of cancer in a subject, e.g., a subject in need thereof, by administering a UBE2K inhibitor (e.g. a specific inhibitor of UBE2K) to said subject.

In some embodiments, the disclosure provides methods of reducing proliferation of a cancer cell, the method comprising contacting the cancer cell with a UBE2K inhibitor (e.g. a specific inhibitor of UBE2K), thereby reducing proliferation of the cancer cell. In some embodiments, the disclosure provides methods of reducing proliferation of a cancer cell, the method comprising administering a UBE2K inhibitor (e.g. a specific inhibitor of UBE2K) to a cancer cell, thereby reducing proliferation of the cancer cell, e.g., relative to a cancer cell that is not treated with the UBE2K inhibitor. In some embodiments, the disclosure provides methods of reducing proliferation of a cancer cell in a subject, e.g. a subject in need thereof, the method comprising administering a UBE2K inhibitor (e.g. a specific inhibitor of UBE2K) to the subject, thereby reducing proliferation of the cancer cell in the subject, e.g., relative to a cancer cell in a subject that is not administered the UBE2K inhibitor.

In some embodiments, the disclosure provides methods of inducing death of a cancer cell, the method comprising contacting the cancer cell with a UBE2K inhibitor (e.g. a specific inhibitor of UBE2K), thereby inducing death of the cancer cell. In some embodiments, the disclosure provides methods of inducing death of a cancer cell, the method comprising administering to the cancer cell a UBE2K inhibitor (e.g. a specific inhibitor of UBE2K), thereby inducing death of the cancer cell. In some embodiments, the disclosure provides methods of inducing death of a cancer cell in a subject, e.g. a subject in need thereof, the method comprising administering to the subject a UBE2K inhibitor (e.g. a specific inhibitor of UBE2K), thereby inducing death of the cancer cell.

In certain embodiments, the cancer comprises a solid tumor. In certain embodiments, the cancer comprises a leukemia. In certain embodiments, the cancer is treated with the UBE2K inhibitor alone. In certain embodiments, the cancer is treated with the UBE2K inhibitor and an additional agent. In certain embodiments, the additional agent is a chemotherapeutic agent.

In one embodiment, administration of the UBE2K inhibitor (e.g. a specific inhibitor of UBE2K) results in one or more of, reducing tumor size, weight or volume, increasing time to progression, inhibiting tumor growth and/or prolonging the survival time of a subject having an oncological disorder. In certain embodiments, administration of the UBE2K inhibitor reduces tumor size, weight or volume, increases time to progression, inhibits tumor growth and/or prolongs the survival time of the subject by at least 1%, 2%, 3%, 4%, 5%, 10%, 20%, 30%, 40%, 50%, 60%, 70%, 80%, 90%, 100%, 200%, 300%, 400% or 500% relative to a corresponding control subject that is not administered the UBE2K inhibitor. In some embodiments, administration of the UBE2K inhibitor stabilizes the cancer in a subject that had a progressive oncological disorder prior to treatment.

In the treatment of cancer, the formulations may be in a pharmaceutically acceptable carrier that may be administered in a therapeutically effective amount to a subject as either a mono-therapy, in combination with at least one other anticancer agent, e.g., chemotherapeutic agent, for a given indication, in combination with radiotherapy, following surgical intervention to radically remove a tumor, in combination with other alternative and/or complementary acceptable treatments for cancer, and the like.

In general, the UBE2K inhibitor (e.g. specific inhibitor of UBE2K) formulations and methods described herein may be used to treat any neoplasm. In a particular embodiment, the formulations and methods are used to treat a solid tumor in a subject. In certain embodiment, the formulations and methods are used to treat a non-solid tumor in a subject, e.g., a leukemia.

In one embodiment, administration of the UBE2K inhibitor (e.g. a specific inhibitor of UBE2K) as described herein, achieves at least stable disease, reduces tumor size, inhibits tumor growth and/or prolongs the survival time of a tumor-bearing subject as compared to an appropriate control. Accordingly, this invention also relates to a method of treating tumors in a human or other animal, by administering to such human or animal an effective, non-toxic amount of a UBE2K inhibitor. One skilled in the art would be able, by routine experimentation with the guidance provided herein, to determine what an effective, non-toxic amount of a UBE2K inhibitor would be for the purpose of treating cancer. For example, a therapeutically active amount of UBE2K inhibitor may vary according to factors such as the disease stage (e.g., stage I versus stage IV), age, sex, medical complications (e.g., immunosuppressed conditions or diseases, coagulopathies) and weight of the subject, and the ability of the UBE2K inhibitor to elicit a desired response in the subject. The dosage regimen may be adjusted to provide the optimum therapeutic response. For example, several divided doses may be administered daily, or the dose may be proportionally reduced as indicated by the exigencies of the therapeutic situation.

In certain embodiments of the invention, the methods further include a treatment regimen which includes any one of or a combination of surgery, radiation, chemotherapy, e.g., hormone therapy, antibody therapy, therapy with growth factors, cytokines, and anti-angiogenic therapy.

Cancers for treatment using the methods of the invention include, for example, all types of cancer or neoplasm or malignant tumors found in mammals, including, but not limited to: leukemias, lymphomas, melanomas, carcinomas and sarcomas. In one embodiment, cancers for treatment using the methods of the invention include melanomas, carcinomas and sarcomas. In preferred embodiments, the UBE2K inhibitor compositions are used for treatment, of various types of solid tumors, for example breast cancer, bladder cancer, colon and rectal cancer, endometrial cancer, kidney (renal cell) cancer, lung cancer, melanoma, pancreatic cancer, prostate cancer, thyroid cancer, skin cancer, bone cancer, brain cancer, cervical cancer, liver cancer, stomach cancer, mouth and oral cancers, neuroblastoma, testicular cancer, uterine cancer, thyroid cancer, head and neck, kidney, lung, non-small cell lung, melanoma, mesothelioma, ovary, sarcoma, stomach, uterus and medulloblastoma, and vulvar cancer. In certain embodiments, solid tumors include breast cancer, including triple negative breast cancer. In certain embodiments, skin cancer includes melanoma, squamous cell carcinoma, cutaneous T-cell lymphoma (CTCL). In certain embodiments, the cancer includes leukemia. In certain embodiments, leukemias include acute leukemias. In certain embodiments, leukemias include chronic leukemias. In certain embodiments, leukemias include acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia. However, treatment using the UBE2K inhibitor compositions are not limited to these types of cancers.

In some embodiments, the cancers for treatment with a UBE2K inhibitor are selected from liver cancer and pancreatic cancer. In some embodiments, the cancers for treatment with a UBE2K inhibitor are selected from choriocarcinoma, ovarian cancer, leukemia (e.g. T cell leukemia, T lymphoblast leukemia, and chronic myelogenous leukemia), lymphoma (e.g. B cell lymphoma and anaplastic large cell lymphoma), embryonic carcinoma, osteosarcoma, skin carcinoma, and colon cancer (e.g. colorectal adenocarcinoma).

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. Leukemia is clinically detectable using one or more of complete blood counts, pallor, blood smears, and bone marrow smears. Advanced leukemias in certain subjects can manifest solid tumors.

Examples of non-solid tumors, e.g., leukemias, that cannot be detected by imaging or palpation can be detected, for example, by neutrophil counts, platelet counts, and by detection of abnormal cells in the bone marrow, e.g., the presence of blasts that cannot be other wise explained (e.g., bone marrow regeneration after consolidation therapy), the presence of Auer rods, or the appearance of new dysplastic changes.

The term “sarcoma” generally refers to a tumor which is made up of a substance like the embryonic connective tissue and is generally composed of closely packed cells embedded in a fibrillar or homogeneous substance. Examples of sarcomas which can be treated with the present compositions and optionally an additional anticancer agent, e.g., a chemotherapeutic agent, include, but are not limited to a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with the compositions of the invention and optionally an additional anticancer agent, e.g., a chemotherapeutic agent, include but are not limited to, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated with the compositions of the invention and optionally an additional anticancer agent, e.g., a chemotherapeutic agent, include but are not limited to, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “leukemia” refers to a type of cancer of the blood or bone marrow characterized by an abnormal increase of immature white blood cells called “blasts”. Leukemia is a broad term covering a spectrum of diseases. In turn, it is part of the even broader group of diseases affecting the blood, bone marrow, and lymphoid system, which are all known as hematological neoplasms. Leukemias can be divided into four major classifications, acute lymphocytic (or lymphoblastic) leukemia (ALL), acute myelogenous (or myeloid or non-lymphatic) leukemia (AML), chronic lymphocytic leukemia (CLL), and chronic myelogenous leukemia (CML). Further types of leukemia include Hairy cell leukemia (HCL), T-cell prolymphocytic leukemia (T-PLL), large granular lymphocytic leukemia, and adult T-cell leukemia.

“Acute leukemia” is characterized by a rapid increase in the number of immature blood cells. Crowding due to such cells makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children.

“Chronic leukemia” is characterized by the excessive build up of relatively mature, but still abnormal, white blood cells. Typically taking months or years to progress, the cells are produced at a much higher rate than normal, resulting in many abnormal white blood cells. Whereas acute leukemia must be treated immediately, chronic forms are sometimes monitored for some time before treatment to ensure maximum effectiveness of therapy.

Lymphoblastic or lymphocytic leukemias are due to hyperproliferation of bone marrow cells that produce lymphocytes (white blood cells), typically B cells.

Myeloid or myelogenous leukemias are due to hyperproliferation of bone marrow cells that produce red blood cells, some other types of white cells, and platelets.

Additional cancers which can be treated with the compounds disclosed herein include, for example, Hodgkin's disease, Non-Hodgkin's lymphoma, multiple myeloma, neuroblastoma, breast cancer, ovarian cancer, lung cancer, rhabdomyo sarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, prostate cancer, pancreatic cancer, uterine sarcoma, myxoid liposarcoma, leiomyosarcoma, chondrosarcoma, osteosarcoma, colon adenocarcinoma of colon, cervical squamous cell carcinoma, tonsil squamous cell carcinoma, papillary thyroid cancer, adenoid cystic cancer, synovial cell sarcoma, malignant fibrous histiocytoma, desmoplastic sarcoma, hepatocellular carcinoma, spindle cell sarcoma, cholangiocarcinoma, and triple negative breast cancer.

In some embodiments, the cancer is not lymphoma. In some embodiments, the cancer is not a B-cell lymphoma. In some embodiments, the cancer is not a lymphoma carrying a MyD88 mutation.

### V. Combination Therapies

The methods of treatment of cancer provided herein include combination therapies with additional anticancer agents or interventions (e.g., radiation, surgery, bone marrow transplant). In certain embodiments, “combination therapy” includes a treatment with UBE2K inhibitor (e.g. a specific inhibitor of UBE2K) to decrease tumor burden and/or improve clinical response. Administration of UBE2K inhibitor with palliative treatments or treatments to mitigate drug side effects (e.g., to decrease nausea, pain, anxiety, or inflammation, to normalize clotting) is not considered to be a combination treatment of the cancer.

In a preferred embodiment, treatment with a UBE2K inhibitor (e.g. a specific inhibitor of UBE2K) is combined with the standard of care for treatment of the particular cancer to be treated. The standard of care for a particular cancer type can be determined by one of skill in the art based on, for example, the type and severity of the cancer, the age, weight, gender, and/or medical history of the subject, and the success or failure of prior treatments.

In certain embodiments, treatment of subjects with solid tumors by a UBE2K inhibitor is combined with one or more of the following treatments.
1. Gemcitabine, preferably by intravenous administration at a weekly dose starting at 600 mg/m², with the dose being adjusted based on the tolerance of the subject to the drug.
2. 5-Fluorouracil (5-FU), preferably by intravenous administration at a weekly starting dose of 350 mg/m², with the dose being adjusted based on the tolerance of the subject to the drug, in combination with leucovorin at 100 mg/m².
3. Docetaxel, preferably by intravenous administration once weekly at a starting dose of 20 mg/m², with the dose being adjusted based on the tolerance of the subject to the drug.

In certain embodiments, 1, 2, 3, 4, or 5 cycles of the combination therapy are administered to the subject. The subject is assessed for response criteria at the end of each cycle. The subject is also monitored throughout each cycle for adverse events (e.g., clotting, anemia, liver and kidney function, etc.) to ensure that the treatment regimen is being sufficiently tolerated.

In certain embodiments, the UBE2K inhibitor (e.g. a specific inhibitor of UBE2K) is administered in an amount that would be therapeutically effective if delivered alone, i.e., UBE2K inhibitor is administered and/or acts as a therapeutic agent, and not predominantly as an agent to ameliorate side effects of other chemotherapy or other cancer treatments. A UBE2K inhibitor and/or pharmaceutical formulations thereof and the other therapeutic agent can act additively or, more preferably, synergistically. In one embodiment, UBE2K inhibitor (e.g. a specific inhibitor of UBE2K) and/or a formulation thereof is administered concurrently with the administration of an additional anticancer (e.g., chemotherapeutic, anti-angiogenic) agent. In another embodiment, a compound and/or pharmaceutical formulation thereof is administered prior or subsequent to administration of another anticancer agent wherein both agents are present in the subject at the same time or have therapeutic activity in the subject at the same time. In one embodiment, the UBE2K inhibitor and additional anticancer agent act synergistically. In one embodiment, the UBE2K inhibitor and additional anticancer agent act additively.

In one embodiment, the therapeutic methods of the invention further comprise administration of one or more additional therapeutic agents, e.g., one or more anticancer agents, e.g., anti-angiogenic agents, chemotherapeutic agents, e.g., small molecule anticancer agents, biologic anticancer agents including both protein based and nucleic acid based therapeutics. For example, in one embodiment, an additional anticancer agent for use in the therapeutic methods of the invention is a chemotherapeutic agent.

Small molecule chemotherapeutic agents generally belong to various classes including, for example: 1. Topoisomerase II inhibitors (cytotoxic antibiotics), such as the anthracyclines/anthracenediones, e.g., doxorubicin, epirubicin, idarubicin and nemorubicin, the anthraquinones, e.g., mitoxantrone and losoxantrone, and the podophillotoxines, e.g., etoposide and teniposide; 2. Agents that affect microtubule formation (mitotic inhibitors), such as plant alkaloids (e.g., a compound belonging to a family of alkaline, nitrogen-containing molecules derived from plants that are biologically active and cytotoxic), e.g., taxanes, e.g., paclitaxel and docetaxel, and the vinka alkaloids, e.g., vinblastine, vincristine, and vinorelbine, and derivatives of podophyllotoxin; 3. Alkylating agents, such as nitrogen mustards, ethyleneimine compounds, alkyl sulphonates and other compounds with an alkylating action such as nitrosoureas, dacarbazine, cyclophosphamide, ifosfamide and melphalan; 4. Antimetabolites (nucleoside inhibitors), for example, folates, e.g., folic acid, fiuropyrimidines, purine or pyrimidine analogues such as 5-fluorouracil, capecitabine, gemcitabine, methotrexate, and edatrexate; 5. Topoisomerase I inhibitors, such as topotecan, irinotecan, and 9-nitrocamptothecin, camptothecin derivatives, and retinoic acid; and 6. Platinum compounds/complexes, such as cisplatin, oxaliplatin, and carboplatin; Exemplary chemotherapeutic agents for use in the methods of the invention include, but are not limited to, amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-I 1, 1O-hydroxy-7-ethyl-camptothecin (SN38), dacarbazine, S-I capecitabine, ftorafur, 5′deoxyflurouridine, UFT, eniluracil, deoxycytidine, 5-azacytosine, 5-azadeoxycytosine, allopurinol, 2-chloro adenosine, trimetrexate, aminopterin, methylene-10-deazaaminopterin (MDAM), oxaplatin, picoplatin, tetraplatin, satraplatin, platinum-DACH, ormaplatin, CI-973, JM-216, and analogs thereof, epirubicin, etoposide phosphate, 9-aminocamptothecin, 10, 11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, Capecitabine, Pentostatin, Trimetrexate, Cladribine, floxuridine, fludarabine, hydroxyurea, ifosfamide, idarubicin, mesna, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, cisplatin, doxorubicin, paclitaxel (taxol), bleomycin, mTor, epidermal growth factor receptor (EGFR), and fibroblast growth factors (FGF) and combinations thereof which are readily apparent to one of skill in the art based on the appropriate standard of care for a particular tumor or cancer.

In another embodiment, an additional chemotherapeutic agent for use in the combination therapies of the invention is a biologic agent. Biologic agents (also called biologics) are the products of a biological system, e.g., an organism, cell, or recombinant system. Examples of such biologic agents include nucleic acid molecules (e.g., antisense nucleic acid molecules), interferons, interleukins, colony-stimulating factors, antibodies, e.g., monoclonal antibodies, anti-angiogenesis agents, and cytokines. Exemplary biologic agents are discussed in more detail below and generally belong to various classes including, for example: 1. Hormones, hormonal analogues, and hormonal complexes, e.g., estrogens and estrogen analogs, progesterone, progesterone analogs and progestins, androgens, adrenocorticosteroids, antiestrogens, antiandrogens, antitestosterones, adrenal steroid inhibitors, and anti-leuteinizing hormones; and 2. Enzymes, proteins, peptides, polyclonal and/or monoclonal antibodies, such as interleukins, interferons, colony stimulating factor, etc.

In one embodiment, the biologic is an interferon. Interferons (IFN) are a type biologic agent that naturally occurs in the body. Interferons are also produced in the laboratory and given to cancer patients in biological therapy. They have been shown to improve the way a cancer patient's immune system acts against cancer cells.

Interferons may work directly on cancer cells to slow their growth, or they may cause cancer cells to change into cells with more normal behavior. Some interferons may also stimulate natural killer cells (NK) cells, T cells, and macrophages which are types of white blood cells in the bloodstream that help to fight cancer cells.

In one embodiment, the biologic is an interleukin. Interleukins (IL) stimulate the growth and activity of many immune cells. They are proteins (cytokines and chemokines) that occur naturally in the body, but can also be made in the laboratory. Some interleukins stimulate the growth and activity of immune cells, such as lymphocytes, which work to destroy cancer cells.

In another embodiment, the biologic is a colony-stimulating factor. Colony-stimulating factors (CSFs) are proteins given to patients to encourage stem cells within the bone marrow to produce more blood cells. The body constantly needs new white blood cells, red blood cells, and platelets, especially when cancer is present. CSFs are given, along with chemotherapy, to help boost the immune system. When cancer patients receive chemotherapy, the bone marrow's ability to produce new blood cells is suppressed, making patients more prone to developing infections. Parts of the immune system cannot function without blood cells, thus colony-stimulating factors encourage the bone marrow stem cells to produce white blood cells, platelets, and red blood cells.

With proper cell production, other cancer treatments can continue enabling patients to safely receive higher doses of chemotherapy.

In another embodiment, the biologic is an antibody. Antibodies, e.g., monoclonal antibodies, are agents, produced in the laboratory, that bind to cancer cells.

Monoclonal antibody agents do not destroy healthy cells. Monoclonal antibodies achieve their therapeutic effect through various mechanisms. They can have direct effects in producing apoptosis or programmed cell death. They can block growth factor receptors, effectively arresting proliferation of tumor cells. In cells that express monoclonal antibodies, they can bring about anti-idiotype antibody formation.

Examples of antibodies which may be used in the combination treatment of the invention include anti-CD20 antibodies, such as, but not limited to, cetuximab, Tositumomab, rituximab, and Ibritumomab. Anti-HER2 antibodies may also be used in combination with UBE2K inhibitor for the treatment of cancer. In one embodiment, the anti-HER2 antibody is Trastuzumab (Herceptin). Other examples of antibodies which may be used in combination with UBE2K inhibitor for the treatment of cancer include anti-CD52 antibodies (e.g., Alemtuzumab), anti-CD-22 antibodies (e.g., Epratuzumab), and anti-CD33 antibodies (e.g., Gemtuzumab ozogamicin). Anti-VEGF antibodies may also be used in combination with

UBE2K inhibitor for the treatment of cancer. In one embodiment, the anti-VEGF antibody is bevacizumab. In other embodiments, the biologic agent is an antibody which is an anti-EGFR antibody e.g., cetuximab. Another example is the anti-glycoprotein 17-1A antibody edrecolomab. Numerous other anti-tumor antibodies are known in the art and would be understood by the skilled artisan to be encompassed by the present invention.

In another embodiment, the biologic is a cytokine. Cytokine therapy uses proteins (cytokines) to help a subject's immune system recognize and destroy those cells that are cancerous. Cytokines are produced naturally in the body by the immune system, but can also be produced in the laboratory. This therapy is used with advanced melanoma and with adjuvant therapy (therapy given after or in addition to the primary cancer treatment). Cytokine therapy reaches all parts of the body to kill cancer cells and prevent tumors from growing.

In another embodiment, the biologic is a fusion protein. For example, recombinant human Apo2L/TRAIL (GENETECH) may be used in a combination therapy. Apo2/TRAIL is the first dual pro-apoptotic receptor agonist designed to activate both pro-apoptotic receptors DR4 and DR5, which are involved in the regulation of apoptosis (programmed cell death).

In one embodiment, the biologic is a therapeutic nucleic acid molecule. Nucleic acid therapeutics are well known in the art. Nucleic acid therapeutics include both single stranded and double stranded (i.e., nucleic acid therapeutics having a complementary region of at least 15 nucleotides in length) nucleic acids that are complementary to a target sequence in a cell. Therapeutic nucleic acids can be directed against essentially any target nucleic acid sequence in a cell. In certain embodiments, the nucleic acid therapeutic is targeted against a nucleic acid sequence encoding a stimulator of angiogenesis, e.g., VEGF, FGF, or of tumor growth, e.g., EGFR.

Antisense nucleic acid therapeutic agents are single stranded nucleic acid therapeutics, typically about 16 to 30 nucleotides in length, and are complementary to a target nucleic acid sequence in the target cell, either in culture or in an organism.

In another aspect, the agent is a single-stranded antisense RNA molecule. An antisense RNA molecule is complementary to a sequence within the target mRNA. Antisense RNA can inhibit translation in a stoichiometric manner by base pairing to the mRNA and physically obstructing the translation machinery, see Dias, N. et al., (2002) Mol Cancer Ther 1:347-355. The antisense RNA molecule may have about 15-30 nucleotides that are complementary to the target mRNA. Patents directed to antisense nucleic acids, chemical modifications, and therapeutic uses are provided, for example, in U.S. Pat. No. 5,898,031 related to chemically modified RNA-containing therapeutic compounds, and U.S. Pat. No. 6,107,094 related methods of using these compounds as therapeutic agent. U.S. Pat. No. 7,432,250 related to methods of treating patients by administering single-stranded chemically modified RNA-like compounds; and U.S. Pat. No. 7,432,249 related to pharmaceutical compositions containing single-stranded chemically modified RNA-like compounds. U.S. Pat. No. 7,629,321 is related to methods of cleaving target mRNA using a single-stranded oligonucleotide having a plurality RNA nucleosides and at least one chemical modification. The entire contents of each of the patents listed in this paragraph are incorporated herein by reference.

Nucleic acid therapeutic agents for use in the methods of the invention also include double stranded nucleic acid therapeutics. An “RNAi agent,” “double stranded RNAi agent,” double-stranded RNA (dsRNA) molecule, also referred to as “dsRNA agent,” “dsRNA”, “siRNA”, “iRNA agent,” as used interchangeably herein, refers to a complex of ribonucleic acid molecules, having a duplex structure comprising two anti-parallel and substantially complementary, as defined below, nucleic acid strands. As used herein, an RNAi agent can also include dsiRNA (see, e.g., US Patent publication 20070104688, incorporated herein by reference). In general, the majority of nucleotides of each strand are ribonucleotides, but as described herein, each or both strands can also include one or more non-ribonucleotides, e.g., a deoxyribonucleotide and/or a modified nucleotide. In addition, as used in this specification, an “RNAi agent” may include ribonucleotides with chemical modifications; an RNAi agent may include substantial modifications at multiple nucleotides. Such modifications may include all types of modifications disclosed herein or known in the art. Any such modifications, as used in a siRNA type molecule, are encompassed by “RNAi agent” for the purposes of this specification and claims. The RNAi agents that are used in the methods of the invention include agents with chemical modifications as disclosed, for example, in U.S. Provisional Application No. 61/561,710, filed on Nov. 18, 2011, International Application No. PCT/US2011/051597, filed on Sep. 15, 2010, and PCT Publication WO 2009/073809, the entire contents of each of which are incorporated herein by reference.

Additional exemplary biologic agents for use in the methods of the invention include, but are not limited to, gefitinib (Iressa), anastrazole, diethylstilbesterol, estradiol, premarin, raloxifene, progesterone, norethynodrel, esthisterone, dimesthisterone, megestrol acetate, medroxyprogesterone acetate, hydroxyprogesterone caproate, norethisterone, methyltestosterone, testosterone, dexamthasone, prednisone, Cortisol, solumedrol, tamoxifen, fulvestrant, toremifene, aminoglutethimide, testolactone, droloxifene, anastrozole, bicalutamide, flutamide, nilutamide, goserelin, flutamide, leuprolide, triptorelin, aminoglutethimide, mitotane, goserelin, cetuximab, erlotinib, imatinib, Tositumomab, Alemtuzumab, Trastuzumab, Gemtuzumab, Rituximab, Ibritumomab tiuxetan, Bevacizumab, Denileukin diftitox, Daclizumab, interferon alpha, interferon beta, anti-4-1BB, anti-4-1BBL, anti-CD40, anti-CD 154, anti-OX40, anti-OX40L, anti-CD28, anti-CD80, anti-CD86, anti-CD70, anti-CD27, anti-HVEM, anti-LIGHT, anti-GITR, anti-GITRL, anti-CTLA-4, soluble OX40L, soluble 4-IBBL, soluble CD154, soluble GITRL, soluble LIGHT, soluble CD70, soluble CD80, soluble CD86, soluble CTLA4-Ig, GVAX®, and combinations thereof which are readily apparent to one of skill in the art based on the appropriate standard of care for a particular tumor or cancer. The soluble forms of agents may be made as, for example fusion proteins, by operatively linking the agent with, for example, Ig-Fc region.

### Immune Checkpoint Modulators

In some embodiments, the additional agent is an immunotherapeutic. In some embodiments, the immunotherapeutic is an immune checkpoint modulator of an immune checkpoint molecule. Examples include LAG-3 (Triebel et al., 1990, J. Exp. Med. 171: 1393-1405), TIM-3 (Sakuishi et al., 2010, J. Exp. Med. 207: 2187-2194) and VISTA (Wang et al., 2011, J. Exp. Med. 208: 577-592). Examples of co-stimulatory molecules that improve immune responses include ICOS (Fan et al., 2014, J. Exp. Med. 211: 715-725), OX40 (Curti et al., 2013, Cancer Res. 73: 7189-7198) and 4-1BB (Melero et al., 1997, Nat. Med. 3: 682-685).

Immune checkpoints may be stimulatory immune checkpoints (i.e. molecules that stimulate the immune response) or inhibitory immune checkpoints (i.e. molecules that inhibit immune response). In some embodiments, the immune checkpoint modulator is an antagonist of an inhibitory immune checkpoint. In some embodiments, the immune checkpoint modulator is an agonist of a stimulatory immune checkpoint. In some embodiments, the immune checkpoint modulator is an immune checkpoint binding protein (e.g., an antibody, antibody Fab fragment, divalent antibody, antibody drug conjugate, scFv, fusion protein, bivalent antibody, or tetravalent antibody). In certain embodiments, the immune checkpoint modulator is capable of binding to, or modulating the activity of more than one immune checkpoint. Examples of stimulatory and inhibitory immune checkpoints, and molecules that modulate these immune checkpoints that may be used in the methods of the invention, are provided below.

### i. Stimulatory Immune Checkpoint Molecules

CD27 supports antigen-specific expansion of naïve T cells and is vital for the generation of T cell memory (see, e.g., Hendriks et al. (2000) Nat. Immunol. 171 (5): 433-40). CD27 is also a memory marker of B cells (see, e.g., Agematsu et al. (2000) Histol. Histopathol. 15 (2): 573-6. CD27 activity is governed by the transient availability of its ligand, CD70, on lymphocytes and dendritic cells (see, e.g., Borst et al. (2005) Curr. Opin. Immunol. 17 (3): 275-81). Multiple immune checkpoint modulators specific for CD27 have been developed and may be used as disclosed herein. In some embodiments, the immune checkpoint modulator is an agent that modulates the activity and/or expression of CD27. In some embodiments, the immune checkpoint modulator is an agent that binds to CD27 (e.g., an anti-CD27 antibody). In some embodiments, the checkpoint modulator is a CD27 agonist. In some embodiments, the checkpoint modulator is a CD27 antagonist. In some embodiments, the immune checkpoint modulator is an CD27-binding protein (e.g., an antibody). In some embodiments, the immune checkpoint modulator is varlilumab (Celldex Therapeutics). Additional CD27-binding proteins (e.g., antibodies) are known in the art and are disclosed, e.g., in U.S. Pat. Nos. 9,248,183, 9,102,737, 9,169,325, 9,023,999, 8,481,029; U.S. Patent Application Publication Nos. 2016/0185870, 2015/0337047, 2015/0299330, 2014/0112942, 2013/0336976, 2013/0243795, 2013/0183316, 2012/0213771, 2012/0093805, 2011/0274685, 2010/0173324; and PCT Publication Nos. WO 2015/016718, WO 2014/140374, WO 2013/138586, WO 2012/004367, WO 2011/130434, WO 2010/001908, and WO 2008/051424, each of which is incorporated by reference herein.

CD28. Cluster of Differentiation 28 (CD28) is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular). Binding with its two ligands, CD80 and CD86, expressed on dendritic cells, prompts T cell expansion (see, e.g., Prasad et al. (1994) Proc. Nat'l. Acad. Sci. USA 91 (7): 2834-8). Multiple immune checkpoint modulators specific for CD28 have been developed and may be used as disclosed herein. In some embodiments, the immune checkpoint modulator is an agent that modulates the activity and/or expression of CD28. In some embodiments, the immune checkpoint modulator is an agent that binds to CD28 (e.g., an anti-CD28 antibody). In some embodiments, the checkpoint modulator is an CD28 agonist. In some embodiments, the checkpoint modulator is an CD28 antagonist. In some embodiments, the immune checkpoint modulator is an CD28-binding protein (e.g., an antibody). In some embodiments, the immune checkpoint modulator is selected from the group consisting of TABO8 (TheraMab LLC), lulizumab (also known as BMS-931699, Bristol-Myers Squibb), and FR104 (OSE Immunotherapeutics). Additional CD28-binding proteins (e.g., antibodies) are known in the art and are disclosed, e.g., in U.S. Pat. Nos. 9,119,840, 8,709,414, 9,085,629, 8,034,585, 7,939,638, 8,389,016, 7,585,960, 8,454,959, 8,168,759, 8,785,604, 7,723,482; U.S. Patent Application Publication Nos. 2016/0017039, 2015/0299321, 2015/0150968, 2015/0071916, 2015/0376278, 2013/0078257, 2013/0230540, 2013/0078236, 2013/0109846, 2013/0266577, 2012/0201814, 2012/0082683, 2012/0219553, 2011/0189735, 2011/0097339, 2010/0266605, 2010/0168400, 2009/0246204, 2008/0038273; and PCT Publication Nos. WO 2015198147, WO 2016/05421, WO 2014/1209168, WO 2011/101791, WO 2010/007376, WO 2010/009391, WO 2004/004768, WO 2002/030459, WO 2002/051871, and WO 2002/047721, each of which is incorporated by reference herein.

CD40. Cluster of Differentiation 40 (CD40, also known as TNFRSFS) is found on a variety of immune system cells including antigen presenting cells. CD40L, otherwise known as CD154, is the ligand of CD40 and is transiently expressed on the surface of activated CD4⁺ T cells. CD40 signaling is known to ‘license’ dendritic cells to mature and thereby trigger T-cell activation and differentiation (see, e.g., O'Sullivan et al. (2003) Crit. Rev. Immunol. 23 (1): 83-107. Multiple immune checkpoint modulators specific for CD40 have been developed and may be used as disclosed herein. In some embodiments, the immune checkpoint modulator is an agent that modulates the activity and/or expression of CD40. In some embodiments, the immune checkpoint modulator is an agent that binds to CD40 (e.g., an anti-CD40 antibody). In some embodiments, the checkpoint modulator is a CD40 agonist. In some embodiments, the checkpoint modulator is an CD40 antagonist. In some embodiments, the immune checkpoint modulator is a CD40-binding protein selected from the group consisting of dacetuzumab (Genentech/Seattle Genetics), CP-870,893 (Pfizer), bleselumab (Astellas Pharma), lucatumumab (Novartis), CFZ533 (Novartis; see, e.g., Cordoba et al. (2015) Am. J. Transplant. 15 (11): 2825-36), RG7876 (Genentech Inc.), FFP104 (PanGenetics, B.V.), APX005 (Apexigen), BI 655064 (Boehringer Ingelheim), Chi Lob 7/4 (Cancer Research UK; see, e.g., Johnson et al. (2015) Clin. Cancer Res. 21 (6): 1321-8), ADC-1013 (Biolnvent International), SEA-CD40 (Seattle Genetics), XmAb 5485 (Xencor), PG120 (PanGenetics B.V.), teneliximab (Bristol-Myers Squibb; see, e.g., Thompson et al. (2011) Am. J. Transplant. 11 (5): 947-57), and AKH3 (Biogen; see, e.g., International Publication No. WO 2016/028810). Additional CD40-binding proteins (e.g., antibodies) are known in the art and are disclosed, e.g., in U.S. Pat. Nos. 9,234,044, 9,266,956, 9,109,011, 9,090,696, 9,023,360, 9,023,361, 9,221,913, 8,945,564, 8,926,979, 8,828,396, 8,637,032, 8,277,810, 8,088,383, 7,820,170, 7,790,166, 7,445,780, 7,361,345, 8,961,991, 8,669,352, 8,957,193, 8,778,345, 8,591,900, 8,551,485, 8,492,531, 8,362,210, 8,388,971; U.S. Patent Application Publication Nos. 2016/0045597, 2016/0152713, 2016/0075792, 2015/0299329, 2015/0057437 2015/0315282, 2015/0307616, 2014/0099317, 2014/0179907, 2014/0349395, 2014/0234344, 2014/0348836, 2014/0193405, 2014/0120103, 2014/0105907, 2014/0248266, 2014/0093497, 2014/0010812, 2013/0024956, 2013/0023047, 2013/0315900, 2012/0087927, 2012/0263732, 2012/0301488, 2011/0027276, 2011/0104182, 2010/0234578, 2009/0304687, 2009/0181015, 2009/0130715, 2009/0311254, 2008/0199471, 2008/0085531, 2016/0152721, 2015/0110783, 2015/0086991, 2015/0086559, 2014/0341898, 2014/0205602, 2014/0004131, 2013/0011405, 2012/0121585, 2011/0033456, 2011/0002934, 2010/0172912, 2009/0081242, 2009/0130095, 2008/0254026, 2008/0075727, 2009/0304706, 2009/0202531, 2009/0117111, 2009/0041773, 2008/0274118, 2008/0057070, 2007/0098717, 2007/0218060, 2007/0098718, 2007/0110754; and PCT Publication Nos. WO 2016/069919, WO 2016/023960, WO 2016/023875, WO 2016/028810, WO 2015/134988, WO 2015/091853, WO 2015/091655, WO 2014/065403, WO 2014/070934, WO 2014/065402, WO 2014/207064, WO 2013/034904, WO 2012/125569, WO 2012/149356, WO 2012/111762, WO 2012/145673, WO 2011/123489, WO 2010/123012, WO 2010/104761, WO 2009/094391, WO 2008/091954, WO 2007/129895, WO 2006/128103, WO 2005/063289, WO 2005/063981, WO 2003/040170, WO 2002/011763, WO 2000/075348, WO 2013/164789, WO 2012/075111, WO 2012/065950, WO 2009/062054, WO 2007/124299, WO 2007/053661, WO 2007/053767, WO 2005/044294, WO 2005/044304, WO 2005/044306, WO 2005/044855, WO 2005/044854, WO 2005/044305, WO 2003/045978, WO 2003/029296, WO 2002/028481, WO 2002/028480, WO 2002/028904, WO 2002/028905, WO 2002/088186, and WO 2001/024823, each of which is incorporated by reference herein.

CD122. CD122 is the Interleukin-2 receptor beta sub-unit and is known to increase proliferation of CD8⁺ effector T cells. See, e.g., Boyman et al. (2012) Nat. Rev. Immunol. 12 (3): 180-190. Multiple immune checkpoint modulators specific for CD122 have been developed and may be used as disclosed herein. In some embodiments, the immune checkpoint modulator is an agent that modulates the activity and/or expression of CD122. In some embodiments, the immune checkpoint modulator is an agent that binds to CD122 (e.g., an anti-CD122 antibody). In some embodiments, the checkpoint modulator is an CD122 agonist. In some embodiments, the checkpoint modulator is an CD22 agonist. In some embodiments, the immune checkpoint modulator is humanized MiK-Beta-1 (Roche; see, e.g., Morris et al. (2006) Proc Nat'l. Acad. Sci. USA 103 (2): 401-6, which is incorporated by reference). Additional CD122-binding proteins (e.g., antibodies) are known in the art and are disclosed, e.g., in U.S. Pat. No. 9,028,830, which is incorporated by reference herein.

OX40. The OX40 receptor (also known as CD134) promotes the expansion of effector and memory T cells. OX40 also suppresses the differentiation and activity of T-regulatory cells, and regulates cytokine production (see, e.g., Croft et al. (2009) Immunol. Rev. 229 (1): 173-91). Multiple immune checkpoint modulators specific for OX40 have been developed and may be used as disclosed herein. In some embodiments, the immune checkpoint modulator is an agent that modulates the activity and/or expression of OX40. In some embodiments, the immune checkpoint modulator is an agent that binds to OX40 (e.g., an anti-OX40 antibody). In some embodiments, the checkpoint modulator is an OX40 agonist. In some embodiments, the checkpoint modulator is an OX40 antagonist. In some embodiments, the immune checkpoint modulator is a OX40-binding protein (e.g., an antibody) selected from the group consisting of MEDI6469 (AgonOx/Medimmune), pogalizumab (also known as MOXR0916 and RG7888; Genentech, Inc.), tavolixizumab (also known as MEDI0562; Medimmune), and GSK3174998 (GlaxoSmithKline). Additional OX-40-binding proteins (e.g., antibodies) are known in the art and are disclosed, e.g., in U.S. Pat. Nos. 9,163,085, 9,040,048, 9,006,396, 8,748,585, 8,614,295, 8,551,477, 8,283,450, 7,550,140; U.S. Patent Application Publication Nos. 2016/0068604, 2016/0031974, 2015/0315281, 2015/0132288, 2014/0308276, 2014/0377284, 2014/0044703, 2014/0294824, 2013/0330344, 2013/0280275, 2013/0243772, 2013/0183315, 2012/0269825, 2012/0244076, 2011/0008368, 2011/0123552, 2010/0254978, 2010/0196359, 2006/0281072; and PCT Publication Nos. WO 2014/148895, WO 2013/068563, WO 2013/038191, WO 2013/028231, WO 2010/096418, WO 2007/062245, and WO 2003/106498, each of which is incorporated by reference herein.

GITR. Glucocorticoid-induced TNFR family related gene (GITR) is a member of the tumor necrosis factor receptor (TNFR) superfamily that is constitutively or conditionally expressed on Treg, CD4, and CD8 T cells. GITR is rapidly upregulated on effector T cells following TCR ligation and activation. The human GITR ligand (GITRL) is constitutively expressed on APCs in secondary lymphoid organs and some nonlymphoid tissues. The downstream effect of GITR:GITRL interaction induces attenuation of Treg activity and enhances CD4⁺ T cell activity, resulting in a reversal of Treg-mediated immunosuppression and increased immune stimulation. Multiple immune checkpoint modulators specific for GITR have been developed and may be used as disclosed herein. In some embodiments, the immune checkpoint modulator is an agent that modulates the activity and/or expression of GITR. In some embodiments, the immune checkpoint modulator is an agent that binds to GITR (e.g., an anti-GITR antibody). In some embodiments, the checkpoint modulator is an GITR agonist. In some embodiments, the checkpoint modulator is an GITR antagonist. In some embodiments, the immune checkpoint modulator is a GITR-binding protein (e.g., an antibody) selected from the group consisting of TRX518 (Leap Therapeutics), MK-4166 (Merck & Co.), MEDI-1873 (MedImmune), INCAGN1876 (Agenus/Incyte), and FPA154 (Five Prime Therapeutics). Additional GITR-binding proteins (e.g., antibodies) are known in the art and are disclosed, e.g., in U.S. Pat. Nos. 9,309,321, 9,255,152, 9,255,151, 9,228,016, 9,028,823, 8,709,424, 8,388,967; U.S. Patent Application Publication Nos. 2016/0145342, 2015/0353637, 2015/0064204, 2014/0348841, 2014/0065152, 2014/0072566, 2014/0072565, 2013/0183321, 2013/0108641, 2012/0189639; and PCT Publication Nos. WO 2016/054638, WO 2016/057841, WO 2016/057846, WO 2015/187835, WO 2015/184099, WO 2015/031667, WO 2011/028683, and WO 2004/107618, each of which is incorporated by reference herein.

ICOS. Inducible T-cell costimulator (ICOS, also known as CD278) is expressed on activated T cells. Its ligand is ICOSL, which is expressed mainly on B cells and dendritic cells. ICOS is important in T cell effector function. ICOS expression is up-regulated upon T cell activation (see, e.g., Fan et al. (2014) J. Exp. Med. 211 (4): 715-25). Multiple immune checkpoint modulators specific for ICOS have been developed and may be used as disclosed herein. In some embodiments, the immune checkpoint modulator is an agent that modulates the activity and/or expression of ICOS. In some embodiments, the immune checkpoint modulator is an agent that binds to ICOS (e.g., an anti-ICOS antibody). In some embodiments, the checkpoint modulator is an ICOS agonist. In some embodiments, the checkpoint modulator is an ICOS antagonist. In some embodiments, the immune checkpoint modulator is a ICOS-binding protein (e.g., an antibody) selected from the group consisting of MEDI-570 (also known as JMab-136, Medimmune), GSK3359609 (GlaxoSmithKline/INSERM), and JTX-2011 (Jounce Therapeutics). Additional ICOS-binding proteins (e.g., antibodies) are known in the art and are disclosed, e.g., in U.S. Pat. Nos. 9,376,493, 7,998,478, 7,465,445, 7,465,444; U.S. Patent Application Publication Nos. 2015/0239978, 2012/0039874, 2008/0199466, 2008/0279851; and PCT Publication No. WO 2001/087981, each of which is incorporated by reference herein.

4-1BB. 4-1BB (also known as CD137) is a member of the tumor necrosis factor (TNF) receptor superfamily. 4-1BB (CD137) is a type II transmembrane glycoprotein that is inducibly expressed on primed CD4⁺ and CD8⁺ T cells, activated NK cells, DCs, and neutrophils, and acts as a T cell costimulatory molecule when bound to the 4-1BB ligand (4-1BBL) found on activated macrophages, B cells, and DCs. Ligation of the 4-1BB receptor leads to activation of the NF-KB, c-Jun and p38 signaling pathways and has been shown to promote survival of CD8⁺ T cells, specifically, by upregulating expression of the antiapoptotic genes BcL-x(L) and Bf1-1. In this manner, 4-1BB serves to boost or even salvage a suboptimal immune response. Multiple immune checkpoint modulators specific for 4-1BB have been developed and may be used as disclosed herein. In some embodiments, the immune checkpoint modulator is an agent that modulates the activity and/or expression of 4-1BB. In some embodiments, the immune checkpoint modulator is an agent that binds to 4-1BB (e.g., an anti-4-1BB antibody). In some embodiments, the checkpoint modulator is an 4-1BB agonist. In some embodiments, the checkpoint modulator is an 4-1BB antagonist. In some embodiments, the immune checkpoint modulator is a 4-1BB-binding protein is urelumab (also known as BMS-663513; Bristol-Myers Squibb) or utomilumab (Pfizer). In some embodiments, the immune checkpoint modulator is a 4-1BB-binding protein (e.g., an antibody). 4-1BB-binding proteins (e.g., antibodies) are known in the art and are disclosed, e.g., in U.S. Pat. Nos. 9,382,328, 8,716,452, 8,475,790, 8,137,667, 7,829,088, 7,659,384; U.S. Patent Application Publication Nos. 2016/0083474, 2016/0152722, 2014/0193422, 2014/0178368, 2013/0149301, 2012/0237498, 2012/0141494, 2012/0076722, 2011/0177104, 2011/0189189, 2010/0183621, 2009/0068192, 2009/0041763, 2008/0305113, 2008/0008716; and PCT Publication Nos. WO 2016/029073, WO 2015/188047, WO 2015/179236, WO 2015/119923, WO 2012/032433, WO 2012/145183, WO 2011/031063, WO 2010/132389, WO 2010/042433, WO 2006/126835, WO 2005/035584, WO 2004/010947; and Martinez-Forero et al. (2013) J. Immunol. 190 (12): 6694-706, and Dubrot et al. (2010) Cancer Immunol. Immunother. 59 (8): 1223-33, each of which is incorporated by reference herein.

### ii. Inhibitory Immune Checkpoint Molecules

ADORA2A. The adenosine A2A receptor (A2A4) is a member of the G protein-coupled receptor (GPCR) family which possess seven transmembrane alpha helices, and is regarded as an important checkpoint in cancer therapy. A2A receptor can negatively regulate overreactive immune cells (see, e.g., Ohta et al. (2001) Nature 414 (6866): 916-20). Multiple immune checkpoint modulators specific for ADORA2A have been developed and may be used as disclosed herein. In some embodiments, the immune checkpoint modulator is an agent that modulates the activity and/or expression of ADORA2A. In some embodiments, the immune checkpoint modulator is an agent that binds to ADORA2A (e.g., an anti-ADORA2A antibody). In some embodiments, the immune checkpoint modulator is a ADORA2A-binding protein (e.g., an antibody). In some embodiments, the checkpoint modulator is an ADORA2A agonist. In some embodiments, the checkpoint modulator is an ADORA2A antagonist. ADORA2A-binding proteins (e.g., antibodies) are known in the art and are disclosed, e.g., in U.S. Patent Application Publication No. 2014/0322236, which is incorporated by reference herein.

B7-H3. B7-H3 (also known as CD276) belongs to the B7 superfamily, a group of molecules that costimulate or down-modulate T-cell responses. B7-H3 potently and consistently down-modulates human T-cell responses (see, e.g., Leitner et al. (2009) Eur. J. Immunol. 39 (7): 1754-64). Multiple immune checkpoint modulators specific for B7-H3 have been developed and may be used as disclosed herein. In some embodiments, the immune checkpoint modulator is an agent that modulates the activity and/or expression of B7-H3. In some embodiments, the immune checkpoint modulator is an agent that binds to B7-H3 (e.g., an anti-B7-H3 antibody). In some embodiments, the checkpoint modulator is an B7-H3 agonist. In some embodiments, the checkpoint modulator is an B7-H3 antagonist. In some embodiments, the immune checkpoint modulator is an anti-B7-H3-binding protein selected from the group consisting of DS-5573 (Daiichi Sankyo, Inc.), enoblituzumab (MacroGenics,

Inc.), and 8H9 (Sloan Kettering Institute for Cancer Research; see, e.g., Ahmed et al. (2015) J. Biol. Chem. 290 (50): 30018-29). In some embodiments, the immune checkpoint modulator is a B7-H3-binding protein (e.g., an antibody). B7-H3-binding proteins (e.g., antibodies) are known in the art and are disclosed, e.g., in U.S. Pat. No. 9,371,395, 9,150,656, 9,062,110, 8,802,091, 8,501,471, 8,414,892; U.S. Patent Application Publication Nos. 2015/0352224, 2015/0297748, 2015/0259434, 2015/0274838, 2014/032875, 2014/0161814, 2013/0287798, 2013/0078234, 2013/0149236, 2012/02947960, 2010/0143245, 2002/0102264; PCT Publication Nos. WO 2016/106004, WO 2016/033225, WO 2015/181267, WO 2014/057687, WO 2012/147713, WO 2011/109400, WO 2008/116219, WO 2003/075846, WO 2002/032375; and Shi et al. (2016) Mol. Med. Rep. 14 (1): 943-8, each of which is incorporated by reference herein.

B7-H4. B7-H4 (also known as O8E, OV064, and V-set domain-containing T-cell activation inhibitor (VTCN1)), belongs to the B7 superfamily. By arresting cell cycle, B7-H4 ligation of T cells has a profound inhibitory effect on the growth, cytokine secretion, and development of cytotoxicity. Administration of B7-H4Ig into mice impairs antigen-specific

T cell responses, whereas blockade of endogenous B7-H4 by specific monoclonal antibody promotes T cell responses (see, e.g., Sica et al. (2003) Immunity 18 (6): 849-61). Multiple immune checkpoint modulators specific for B7-H4 have been developed and may be used as disclosed herein. In some embodiments, the immune checkpoint modulator is an agent that modulates the activity and/or expression of B7-H4. In some embodiments, the immune checkpoint modulator is an agent that binds to B7-H4 (e.g., an anti-B7-H4 antibody). In some embodiments, the immune checkpoint modulator is a B7-H4-binding protein (e.g., an antibody). In some embodiments, the checkpoint modulator is an B7-H4 agonist. In some embodiments, the checkpoint modulator is an B7-H4 antagonist. B7-H4-binding proteins (e.g., antibodies) are known in the art and are disclosed, e.g., in U.S. Pat. Nos. 9,296,822, 8,609,816, 8,759,490, 8,323,645; U.S. Patent Application Publication Nos. 2016/0159910, 2016/0017040, 2016/0168249, 2015/0315275, 2014/0134180, 2014/0322129, 2014/0356364, 2014/0328751, 2014/0294861, 2014/0308259, 2013/0058864, 2011/0085970, 2009/0074660, 2009/0208489; and PCT Publication Nos. WO 2016/040724, WO 2016/070001, WO 2014/159835, WO 2014/100483, WO 2014/100439, WO 2013/067492, WO 2013/025779, WO 2009/073533, WO 2007/067991, and WO 2006/104677, each of which is incorporated by reference herein.

BTLA. B and T Lymphocyte Attenuator (BTLA), also known as CD272, has HVEM (Herpesvirus Entry Mediator) as its ligand. Surface expression of BTLA is gradually downregulated during differentiation of human CD8⁺ T cells from the naive to effector cell phenotype, however tumor-specific human CD8⁺ T cells express high levels of BTLA (see, e.g., Derre et al. (2010) J. Clin. Invest. 120 (1): 157-67). Multiple immune checkpoint modulators specific for BTLA have been developed and may be used as disclosed herein. In some embodiments, the immune checkpoint modulator is an agent that modulates the activity and/or expression of BTLA. In some embodiments, the immune checkpoint modulator is an agent that binds to BTLA (e.g., an anti-BTLA antibody). In some embodiments, the immune checkpoint modulator is a BTLA-binding protein (e.g., an antibody). In some embodiments, the checkpoint modulator is an BTLA agonist. In some embodiments, the checkpoint modulator is an BTLA antagonist. BTLA-binding proteins (e.g., antibodies) are known in the art and are disclosed, e.g., in U.S. Pat. Nos. 9,346,882, 8,580,259, 8,563,694, 8,247,537; U.S. Patent Application Publication Nos. 2014/0017255, 2012/0288500, 2012/0183565, 2010/0172900; and PCT Publication Nos. WO 2011/014438, and WO 2008/076560, each of which is incorporated by reference herein.

CTLA-4. Cytotoxic T lymphocyte antigen-4 (CTLA-4) is a member of the immune regulatory CD28-B7 immunoglobulin superfamily and acts on naïve and resting T lymphocytes to promote immunosuppression through both B7-dependent and B7-independent pathways (see, e.g., Kim et al. (2016) J. Immunol. Res., Article ID 4683607, 14 pp.). CTLA-4 is also known as called CD152. CTLA-4 modulates the threshold for T cell activation. See, e.g., Gajewski et al. (2001) J. Immunol. 166 (6): 3900-7. Multiple immune checkpoint modulators specific for CTLA-4 have been developed and may be used as disclosed herein. In some embodiments, the immune checkpoint modulator is an agent that modulates the activity and/or expression of CTLA-4. In some embodiments, the immune checkpoint modulator is an agent that binds to CTLA-4 (e.g., an anti-CTLA-4 antibody). In some embodiments, the checkpoint modulator is an CTLA-4 agonist. In some embodiments, the checkpoint modulator is an CTLA-4 antagonist. In some embodiments, the immune checkpoint modulator is a CTLA-4-binding protein (e.g., an antibody) selected from the group consisting of ipilimumab (Yervoy; Medarex/Bristol-Myers Squibb), tremelimumab (formerly ticilimumab; Pfizer/Astra7eneca), JMW-3B3 (University of Aberdeen), and AGEN1884 (Agenus). Additional CTLA-4 binding proteins (e.g., antibodies) are known in the art and are disclosed, e.g., in U.S. Pat. No. 8,697,845; U.S. Patent Application Publication Nos. 2014/0105914, 2013/0267688, 2012/0107320, 2009/0123477; and PCT Publication Nos. WO 2014/207064, WO 2012/120125, WO 2016/015675, WO 2010/097597, WO 2006/066568, and WO 2001/054732, each of which is incorporated by reference herein.

IDO. Indoleamine 2,3-dioxygenase (IDO) is a tryptophan catabolic enzyme with immune-inhibitory properties. Another important molecule is TDO, tryptophan 2,3-dioxygenase. IDO is known to suppress T and NK cells, generate and activate Tregs and myeloid-derived suppressor cells, and promote tumor angiogenesis. Prendergast et al., 2014, Cancer Immunol Immunother. 63 (7): 721-35, which is incorporated by reference herein.

Multiple immune checkpoint modulators specific for IDO have been developed and may be used as disclosed herein. In some embodiments, the immune checkpoint modulator is an agent that modulates the activity and/or expression of IDO. In some embodiments, the immune checkpoint modulator is an agent that binds to IDO (e.g., an IDO binding protein, such as an anti-IDO antibody). In some embodiments, the checkpoint modulator is an IDO agonist. In some embodiments, the checkpoint modulator is an IDO antagonist. In some embodiments, the immune checkpoint modulator is selected from the group consisting of Norharmane, Rosmarinic acid, COX-2 inhibitors, alpha-methyl-tryptophan, and Epacadostat. In one embodiment, the modulator is Epacadostat.

KIR. Killer immunoglobulin-like receptors (KIRs) comprise a diverse repertoire of MHCI binding molecules that negatively regulate natural killer (NK) cell function to protect cells from NK-mediated cell lysis. KIRs are generally expressed on NK cells but have also been detected on tumor specific CTLs. Multiple immune checkpoint modulators specific for MR have been developed and may be used as disclosed herein. In some embodiments, the immune checkpoint modulator is an agent that modulates the activity and/or expression of MR. In some embodiments, the immune checkpoint modulator is an agent that binds to MR (e.g., an anti-MR antibody). In some embodiments, the immune checkpoint modulator is a MR-binding protein (e.g., an antibody). In some embodiments, the checkpoint modulator is an MR agonist. In some embodiments, the checkpoint modulator is an MR antagonist. In some embodiments the immune checkpoint modulator is lirilumab (also known as BMS-986015; Bristol-Myers Squibb). Additional MR binding proteins (e.g., antibodies) are known in the art and are disclosed, e.g., in U.S. Pat. Nos. 8,981,065, 9,018,366, 9,067,997, 8,709,411, 8,637,258, 8,614,307, 8,551,483, 8,388,970, 8,119,775; U.S. Patent Application Publication Nos. 2015/0344576, 2015/0376275, 2016/0046712, 2015/0191547, 2015/0290316, 2015/0283234, 2015/0197569, 2014/0193430, 2013/0143269, 2013/0287770, 2012/0208237, 2011/0293627, 2009/0081240, 2010/0189723; and PCT Publication Nos. WO 2016/069589, WO 2015/069785, WO 2014/066532, WO 2014/055648, WO 2012/160448, WO 2012/071411, WO 2010/065939, WO 2008/084106, WO 2006/072625, WO 2006/072626, and WO 2006/003179, each of which is incorporated by reference herein.

LAG-3, Lymphocyte-activation gene 3 (LAG-3, also known as CD223) is a CD4-related transmembrane protein that competitively binds MHC II and acts as a co-inhibitory checkpoint for T cell activation (see, e.g., Goldberg and Drake (2011) Curr. Top. Microbiol. Immunol. 344: 269-78). Multiple immune checkpoint modulators specific for LAG-3 have been developed and may be used as disclosed herein. In some embodiments, the immune checkpoint modulator is an agent that modulates the activity and/or expression of LAG-3. In some embodiments, the immune checkpoint modulator is an agent that binds to LAG-3 (e.g., an anti-PD-1 antibody). In some embodiments, the checkpoint modulator is an LAG-3 agonist. In some embodiments, the checkpoint modulator is an LAG-3 antagonist. In some embodiments, the immune checkpoint modulator is a LAG-3-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANB011; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-1. Programmed cell death protein 1 (PD-1, also known as CD279 and PDCD1) is an inhibitory receptor that negatively regulates the immune system. In contrast to CTLA-4 which mainly affects naïve T cells, PD-1 is more broadly expressed on immune cells and regulates mature T cell activity in peripheral tissues and in the tumor microenvironment. PD-1 inhibits T cell responses by interfering with T cell receptor signaling. PD-1 has two ligands, PD-L1 and PD-L2. Multiple immune checkpoint modulators specific for PD-1 have been developed and may be used as disclosed herein. In some embodiments, the immune checkpoint modulator is an agent that modulates the activity and/or expression of PD-1. In some embodiments, the immune checkpoint modulator is an agent that binds to PD-1 (e.g., an anti-PD-1 antibody). In some embodiments, the checkpoint modulator is an PD-1 agonist. In some embodiments, the checkpoint modulator is an PD-1 antagonist. In some embodiments, the immune checkpoint modulator is a PD-1-binding protein (e.g., an antibody) selected from the group consisting of pembrolizumab (Keytruda; formerly lambrolizumab; Merck & Co., Inc.), nivolumab (Opdivo; Bristol-Myers Squibb), pidilizumab (CT-011, CureTech), SHR-1210 (Incyte/Jiangsu Hengrui Medicine Co., Ltd.), MEDI0680 (also known as AMP-514; Amplimmune Inc./Medimmune), PDR001 (Novartis), BGB-A317 (BeiGene Ltd.), TSR-042 (also known as ANB011; AnaptysBio/Tesaro, Inc.), REGN2810 (Regeneron Pharmaceuticals, Inc./Sanofi-Aventis), and PF-06801591 (Pfizer). Additional PD-1-binding proteins (e.g., antibodies) are known in the art and are disclosed, e.g., in U.S. Pat. Nos. 9,181,342, 8,927,697, 7,488,802, 7,029,674; U.S. Patent Application Publication Nos. 2015/0152180, 2011/0171215, 2011/0171220; and PCT Publication Nos. WO 2004/056875, WO 2015/036394, WO 2010/029435, WO 2010/029434, WO 2014/194302, each of which is incorporated by reference herein.

PD-L1/PD-L2. PD ligand 1 (PD-L1, also knows as B7-H1) and PD ligand 2 (PD-L2, also known as PDCD1LG2, CD273, and B7-DC) bind to the PD-1 receptor. Both ligands belong to the same B7 family as the B7-1 and B7-2 proteins that interact with CD28 and CTLA-4. PD-L1 can be expressed on many cell types including, for example, epithelial cells, endothelial cells, and immune cells. Ligation of PDL-1 decreases IFNγ, TNFα, and IL-2 production and stimulates production of IL10, an anti-inflammatory cytokine associated with decreased T cell reactivity and proliferation as well as antigen-specific T cell anergy. PDL-2 is predominantly expressed on antigen presenting cells (APCs). PDL2 ligation also results in T cell suppression, but where PDL-1-PD-1 interactions inhibits proliferation via cell cycle arrest in the G1/G2 phase, PDL2-PD-1 engagement has been shown to inhibit TCR-mediated signaling by blocking B7:CD28 signals at low antigen concentrations and reducing cytokine production at high antigen concentrations. Multiple immune checkpoint modulators specific for PD-L1 and PD-L2 have been developed and may be used as disclosed herein.

In some embodiments, the immune checkpoint modulator is an agent that modulates the activity and/or expression of PD-L1. In some embodiments, the immune checkpoint modulator is an agent that binds to PD-L1 (e.g., an anti-PD-L1 antibody). In some embodiments, the checkpoint modulator is an PD-L1 agonist. In some embodiments, the checkpoint modulator is an PD-L1 antagonist. In some embodiments, the immune checkpoint modulator is a PD-L1-binding protein (e.g., an antibody or a Fc-fusion protein) selected from the group consisting of durvalumab (also known as MEDI-4736; Astra7eneca/Celgene Corp./Medimmune), atezolizumab (Tecentriq; also known as MPDL3280A and RG7446; Genetech Inc.), avelumab (also known as MSB0010718C; Merck Serono/Astra7eneca); MDX-1105 (Medarex/Bristol-Meyers Squibb), AMP-224 (Amplimmune, GlaxoSmithKline), LY3300054 (Eli Lilly and Co.). Additional PD-L1-binding proteins are known in the art and are disclosed, e.g., in U.S. Patent Application Publication Nos. 2016/0084839, 2015/0355184, 2016/0175397, and PCT Publication Nos. WO 2014/100079, WO 2016/030350, WO2013181634, each of which is incorporated by reference herein.

In some embodiments, the immune checkpoint modulator is an agent that modulates the activity and/or expression of PD-L2. In some embodiments, the immune checkpoint modulator is an agent that binds to PD-L2 (e.g., an anti-PD-L2 antibody). In some embodiments, the checkpoint modulator is an PD-L2 agonist. In some embodiments, the checkpoint modulator is an PD-L2 antagonist. PD-L2-binding proteins (e.g., antibodies) are known in the art and are disclosed, e.g., in U.S. Pat. Nos. 9,255,147, 8,188,238; U.S. Patent Application Publication Nos. 2016/0122431, 2013/0243752, 2010/0278816, 2016/0137731, 2015/0197571, 2013/0291136, 2011/0271358; and PCT Publication Nos. WO 2014/022758, and WO 2010/036959, each of which is incorporated by reference herein.

TIM-3. T cell immunoglobulin mucin 3 (TIM-3, also known as Hepatitis A virus cellular receptor (HAVCR2)) is a A type I glycoprotein receptor that binds to S-type lectin galectin-9 (Gal-9). TIM-3, is a widely expressed ligand on lymphocytes, liver, small intestine, thymus, kidney, spleen, lung, muscle, reticulocytes, and brain tissue. Tim-3 was originally identified as being selectively expressed on IFN-γ-secreting Th1 and Tc1 cells (Monney et al. (2002) Nature 415: 536-41). Binding of Gal-9 by the TIM-3 receptor triggers downstream signaling to negatively regulate T cell survival and function. Multiple immune checkpoint modulators specific for TIM-3 have been developed and may be used as disclosed herein. In some embodiments, the immune checkpoint modulator is an agent that modulates the activity and/or expression of TIM-3. In some embodiments, the immune checkpoint modulator is an agent that binds to TIM-3 (e.g., an anti-TIM-3 antibody). In some embodiments, the checkpoint modulator is an TIM-3 agonist. In some embodiments, the checkpoint modulator is an TIM-3 antagonist. In some embodiments, the immune checkpoint modulator is an anti-TIM-3 antibody selected from the group consisting of TSR-022 (AnaptysBio/Tesaro, Inc.) and MGB453 (Novartis). Additional TIM-3 binding proteins (e.g., antibodies) are known in the art and are disclosed, e.g., in U.S. Pat. Nos. 9,103,832, 8,552,156, 8,647,623, 8,841,418; U.S. Patent Application Publication Nos. 2016/0200815, 2015/0284468, 2014/0134639, 2014/0044728, 2012/0189617, 2015/0086574, 2013/0022623; and PCT Publication Nos. WO 2016/068802, WO 2016/068803, WO 2016/071448, WO 2011/155607, and WO 2013/006490, each of which is incorporated by reference herein.

VISTA. V-domain Ig suppressor of T cell activation (VISTA, also known as Platelet receptor Gi24) is an Ig super-family ligand that negatively regulates T cell responses. See, e.g., Wang et al., 2011, J. Exp. Med. 208: 577-92. VISTA expressed on APCs directly suppresses CD4⁺ and CD8⁺ T cell proliferation and cytokine production (Wang et al. (2010) J Exp Med. 208 (3): 577-92). Multiple immune checkpoint modulators specific for VISTA have been developed and may be used as disclosed herein. In some embodiments, the immune checkpoint modulator is an agent that modulates the activity and/or expression of VISTA. In some embodiments, the immune checkpoint modulator is an agent that binds to VISTA (e.g., an anti-VISTA antibody). In some embodiments, the checkpoint modulator is an VISTA agonist. In some embodiments, the checkpoint modulator is an VISTA antagonist. In some embodiments, the immune checkpoint modulator is a VISTA-binding protein (e.g., an antibody) selected from the group consisting of TSR-022 (AnaptysBio/Tesaro, Inc.) and MGB453 (Novartis). VISTA-binding proteins (e.g., antibodies) are known in the art and are disclosed, e.g., in U.S. Patent Application Publication Nos. 2016/0096891, 2016/0096891; and PCT Publication Nos. WO 2014/190356, WO 2014/197849, WO 2014/190356 and WO 2016/094837, each of which is incorporated by reference herein.

In some embodiments, more than one (e.g. 2, 3, 4, 5 or more) immune checkpoint modulator is administered to the subject. Where more than one immune checkpoint modulator is administered, the modulators may each target a stimulatory immune checkpoint molecule, or each target an inhibitory immune checkpoint molecule. In other embodiments, the immune checkpoint modulators include at least one modulator targeting a stimulatory immune checkpoint and at least one immune checkpoint modulator targeting an inhibitory immune checkpoint molecule. In certain embodiments, the immune checkpoint modulator is a binding protein, for example, an antibody. The term “binding protein”, as used herein, refers to a protein or polypeptide that can specifically bind to a target molecule, e.g. an immune checkpoint molecule. In some embodiments the binding protein is an antibody or antigen binding portion thereof, and the target molecule is an immune checkpoint molecule. In some embodiments the binding protein is a protein or polypeptide that specifically binds to a target molecule (e.g., an immune checkpoint molecule). In some embodiments the binding protein is a ligand. In some embodiments, the binding protein is a fusion protein. In some embodiments, the binding protein is a receptor. Examples of binding proteins that may be used in the methods of the invention include, but are not limited to, a humanized antibody, an antibody Fab fragment, a divalent antibody, an antibody drug conjugate, a scFv, a fusion protein, a bivalent antibody, and a tetravalent antibody.

Immune checkpoint modulator antibodies include, but are not limited to, at least 4 major categories: i) antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, and KIR), ii) antibodies that activate stimulatory pathways directly on T cells or NK cells (e.g., antibodies targeting OX40, GITR, and 4-1BB), iii) antibodies that block a suppressive pathway on immune cells or relies on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, and Ly6G), and iv) antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, and MUC1). Examples of checkpoint inhibitors include, e.g., an inhibitor of CTLA-4, such as ipilimumab or tremelimumab; an inhibitor of the PD-1 pathway such as an anti-PD-1, anti-PD-L1 or anti-PD-L2 antibody. Exemplary anti-PD-1 antibodies are described in WO 2006/121168, WO 2008/156712, WO 2012/145493, WO 2009/014708 and WO 2009/114335. Exemplary anti-PD-L1 antibodies are described in WO 2007/005874, WO 2010/077634 and WO 2011/066389, and exemplary anti-PD-L2 antibodies are described in WO 2004/007679.

In a particular embodiment, the immune checkpoint modulator is a fusion protein, for example, a fusion protein that modulates the activity of an immune checkpoint modulator. In one embodiment, the immune checkpoint modulator is a therapeutic nucleic acid molecule, for example a nucleic acid that modulates the expression of an immune checkpoint protein or mRNA. Nucleic acid therapeutics are well known in the art. Nucleic acid therapeutics include both single stranded and double stranded (i.e., nucleic acid therapeutics having a complementary region of at least 15 nucleotides in length) nucleic acids that are complementary to a target sequence in a cell. In certain embodiments, the nucleic acid therapeutic is targeted against a nucleic acid sequence encoding an immune checkpoint protein.

It should be noted that more than one additional anticancer agents, e.g., 2, 3, 4, 5, or more, may be administered in combination with the UBE2K inhibitor (e.g. a specific inhibitor of UBE2K) formulations provided herein. For example, in one embodiment two additional chemotherapeutic agents may be administered in combination with the UBE2K inhibitor. Appropriate doses and routes of administration of the chemotherapeutic agents provided herein are known in the art.

### EXAMPLES

### Example 1

### Description of Identifying a Role for UBE2K in Cancer Using Network Biology

UBE2K (E2-25K) was identified from the first generation cancer network by employing the Interrogative Biology™ platform in an in vitro pan-cancer model. The Interrogative Biology™ platform is described, for example, in WO/2012/119129, which is incorporated by reference herein in its entAirety. The in vitro pan-cancer model consisted of cultured cancer cells representing diverse tissues of origin, i.e. liver (HepG2), pancreas (MIA PaCa2), skin (SKMEL28 melanoma), tongue (SCC-25 squamous cell carcinoma), breast (SkBr-3, MCF7), and prostate (PC-3 and LnCAP), as well as non-tumorigenic cells derived from breast, pancreas, prostate, liver, and dermis. Cells were exposed to in vitro conditions designed to simulate poor oxygenation, low pH, and diminished nutrient microenvironment as follows. Non-tumorigenic cells and cancer cells were cultured in low (5 mM)- or high (22 mM)-glucose, with and without lactic acid (12.5 mM), with and without Coenzyme Q10 (50 and 100 μM) at normoxia (˜21% oxygen) or hypoxia (2% oxygen). Cell lysates were harvested at 24 and 48 h for proteomics analysis, and results were used as inputs for the Bayesian Network Inference model. Prioritization and ranking of candidate oncology targets was performed based on number of in degree and out degree connections (edges) and the frequency/AUC of the connectivity.

### Example 2

### Knockdown of UBE2K in Cancer Cell Lines in Vitro

### Rationale for Cell Line Selection

Given that UBE2K was identified from a pan-cancer in vitro model representative of multiple tissue of origin/cell lines, a subset were selected for the initial in vitro validation studies. Based on the proteomics data used for network generation, robust changes in UBE2K protein expression were identified in the Miapaca2 pancreatic cancer cell line. In addition, because UBE2K can work in concert with two relevant tumor suppressors (p53, BRCA1), we selected additional cell lines representative of tumor types in which these tumor suppressors are relevant and that were included in the pan-cancer in vitro model, namely breast cancer and hepatocellular carcinoma (HCC). SkBr-3 (breast) and HepG2 (HCC) were used in for network generation. T47D, MDA-MB231, and BT549 (breast) as well as SKHEP1 and Hep3B (HCC) were also included to provide more diversity with regard to p53 and BRCA1 status, allowing for the possibility of capturing canonical and non-canonical functions of UBE2K in these contexts. Cell lines used for initial validation studies are summarized in Table 1.

TABLE 1
Cell lines used for initial validation studies

| | | | | | Used for |
|---|---|---|---|---|---|
| Tumor | Cell | | | | network |
| Model | Lines | P53 | BRCA1 | Tumorigenic | generation |
| PDAC | Miapaca2 | Mut/Mut* | | Yes | Yes |
| HCC | HepG2 | WT/WT | | Yes | Yes |
| | SKHEP1 | WT/WT | | Yes | No |
| | Hep3B | —/— | | Yes | No |
| Breast | MDA- | Mut/Mut | WT/allelic | Yes | No |
| | MB231 | | loss | | |
| | T47D | Mut/Mut | WT/no loss | Yes | No |
| | SKBR3 | Mut/Mut* | WT/allelic | Yes | Yes |
| | | | loss | | |
| | BT549 | Mut/Mut* | WT/allelic | Yes | No |
| | | | loss | | |

*p53 hot spot mutation codon

### Initial Validation Strategy

For initial validation studies, UBE2K was transiently knocked down using an siRNA mediated approach in Miapaca2, HepG2, SKHEP1, Hep3B, MDAMB231, SKBR3, T47D and BT549 cells. A non-targeting (NT) siRNA sequence was used as a control. Samples for confirmation of knock down at transcript and protein level were harvested 24 and 96 h post transfection, and the effect of UBE2K transient knock down was assessed on cell viability/cell number using Cell Titer Fluor at 96 h post transfection. In parallel, the effect of siRNA mediated UBE2K knock down on doxorubicin (cell death causing agent) sensitivity was assessed in the above mentioned cell lines. Cell were exposed to doxorubicin for 72 h beginning 24 h post transfection.

### In Vitro Experimental Results

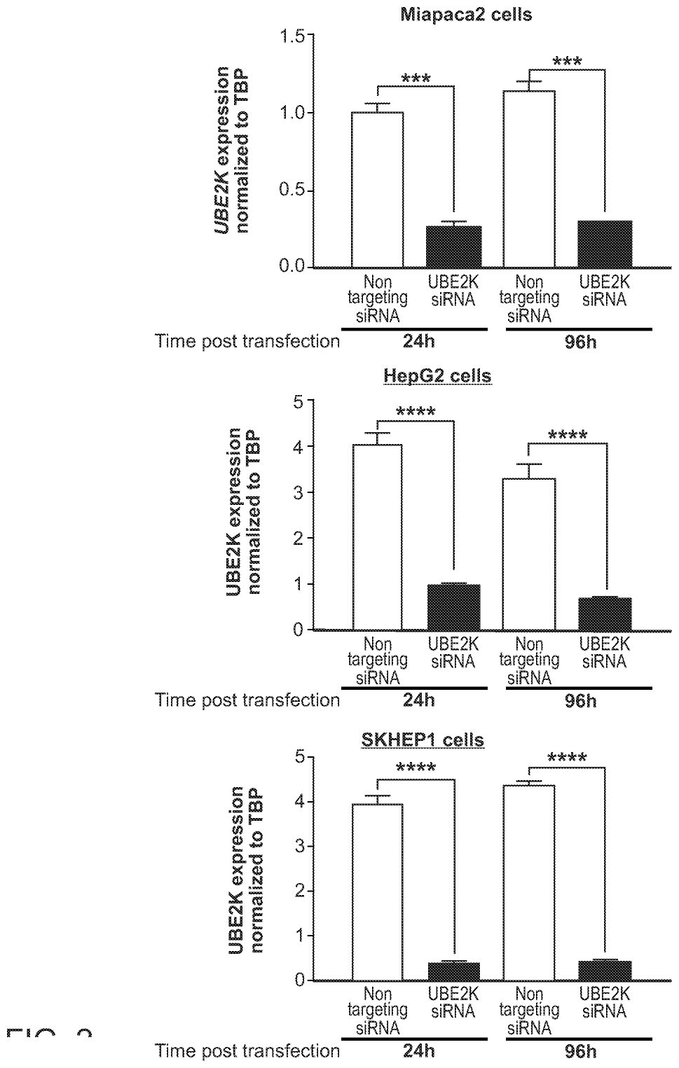

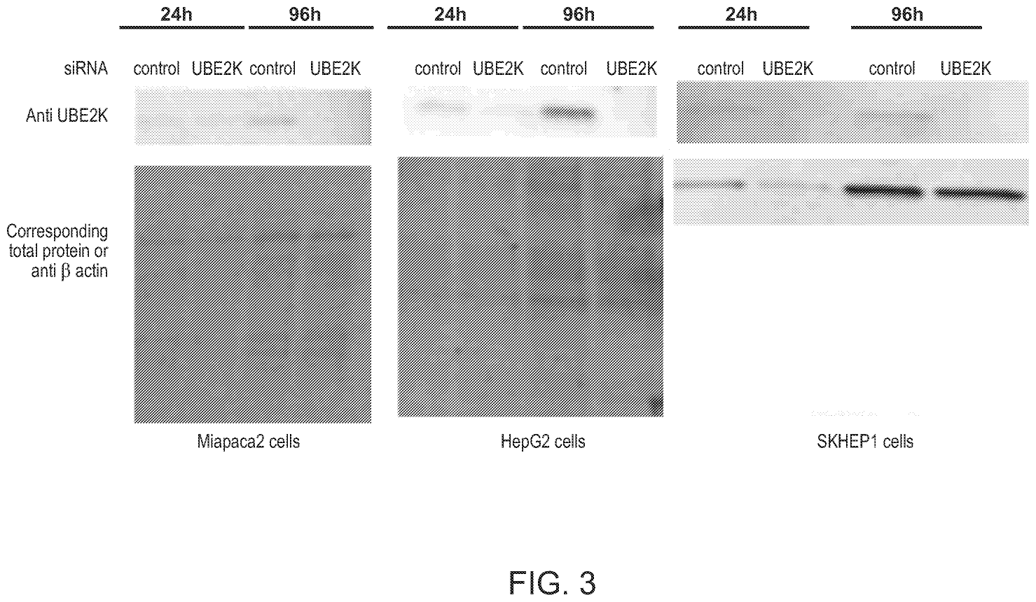

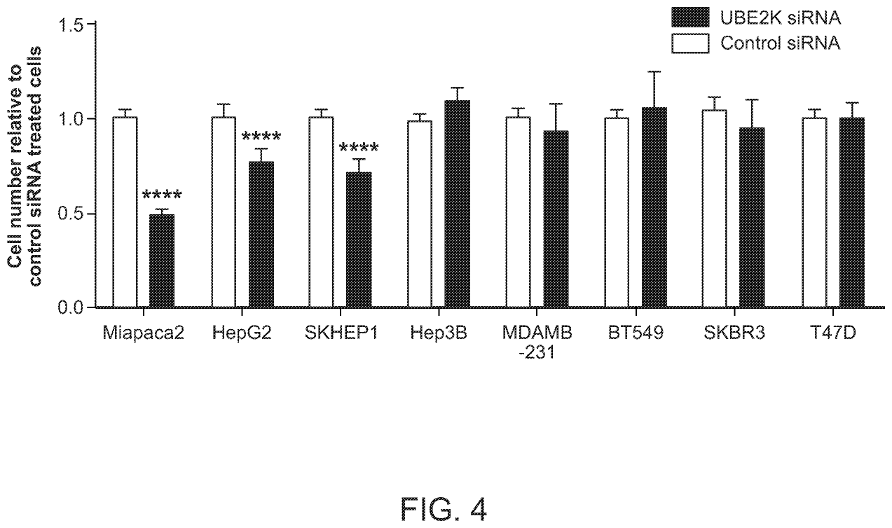

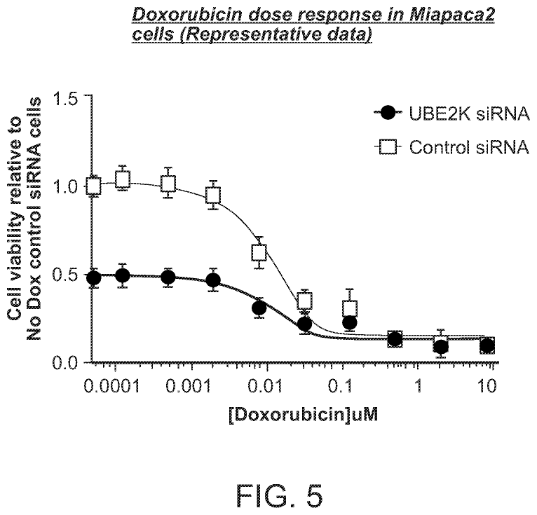

Using this siRNA mediated approach, greater than 70% UBE2K knock down was achieved at 24 h post transfection at both the transcript and protein level in all the tested cell lines (see FIG. 2 and FIG. 3), which provided a reasonable window to assess the effect of UBE2K knock down in phenotypic assays. Under basal conditions, UBE2K siRNA mediated knockdown resulted in a 50% decrease in cell number in Miapaca2 pancreatic cancer cells and a 30% decrease in cell number in SKHEP1 and HepG2 hepatocellular carcinoma cells at 96 h post transfection (FIG. 4). No effect of UBE2K siRNA was observed in the remaining 5 cancer cell lines. In addition, sensitivity to doxorubicin-induced cell death was unaltered in response to UBE2K siRNA mediated knockdown in all the tested cell lines (FIG. 5 and Table 2). Table 3 shows a summary of the results.

TABLE 2
Effect of UBE2K siRNA mediated knockdown on
doxorubicin sensitivity. 95% confidence intervals (CI) for the IC₅₀
values for doxorubicin in cells with and without siRNA-
mediated knockdown of UBE2K are provided. Given the
comparable IC₅₀ values and overlapping 95% confidence intervals,
these data indicate that the presence or absence of
UBE2K does not change the sensitivity to doxorubicin-
induced cell cycle arrest and/or cell death.

| | Control siRNA | UBE2K siRNA |
|---|---|---|
| Cell line | 95% CI KSO (nM) | 95% CI IC₅₀ (nM) |
| Miapaca2 | 9.32 to 14.54 | 10.94 to 27.20 |
| Hep02 | 64.94 to 519.35 | 100.33 to 661.73 |
| SKHEP1 | 8.58 to 11.33 | 10.05 to 13.66 |
| MDAMB-231 | 131.64 to 265.16 | 129.20 to 196.52 |
| T47D | 112.21 to 289.89 | 96.10 to 220.44 |
| BT549 | 165 to 380.89 | 91.16 to 242.53 |
| SKBR3 | 83.04 to 221.25 | 92.40 to 148.05 |

TABLE 3
Summary of the results for the effect of UBE2K siRNA
mediated knock down on cell number and doxorubicin sensitivity.

| | | | | Effect on phenotype | |
|---|---|---|---|---|---|
| | | | | Basal | Stimulated with |
| | | | | conditions | Doxorubicin |
| | | | | (End point | (End point |
| | | | | read: Cell | read: cell |
| | | Confirmation | | number | number |
| Tumor | Cell | of knock down | | using Cell | using Cell |
| model | lines | mRNA | Protein | Titer Fluor) | Titer Fluor) |
| PDAC | Miapaca2 | Yes | Yes | 50% ↓ | No change |
| HCC | HepG2 | Yes | Yes | 30% ↓ | No change |
| | SKHEP1 | Yes | Yes | 30% ↓ | No change |
| | Hep3B | Yes | Yes | No change | No change |
| Breast | MDA- | Yes | Yes | No change | No change |
| | MB231 | | | | |
| | T47D | Yes | Yes | No change | No change |
| | SKBR3 | Yes | Yes | No change | No change |
| | BT549 | Yes | Yes | No change | No change |

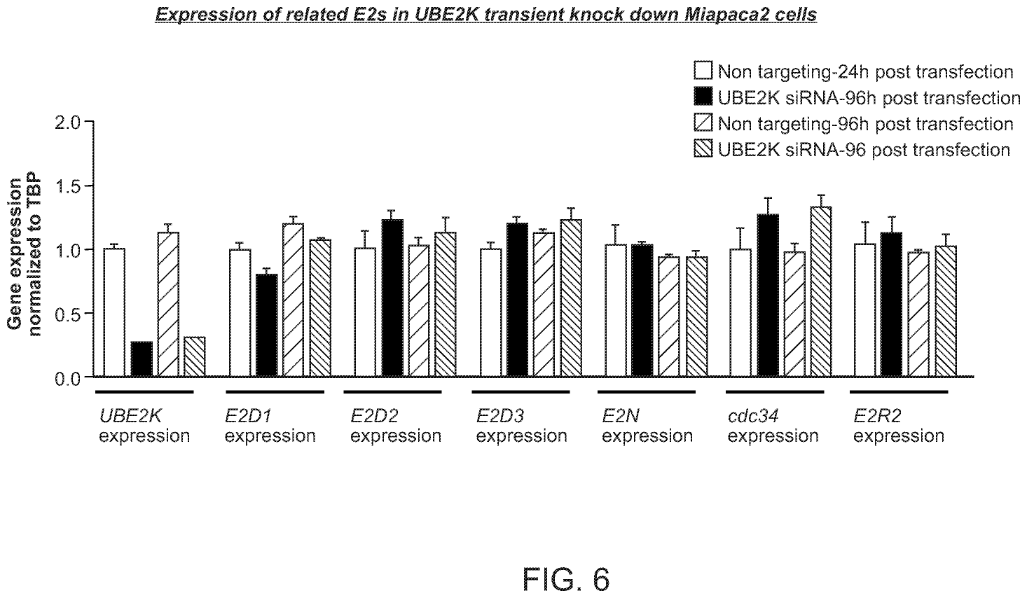

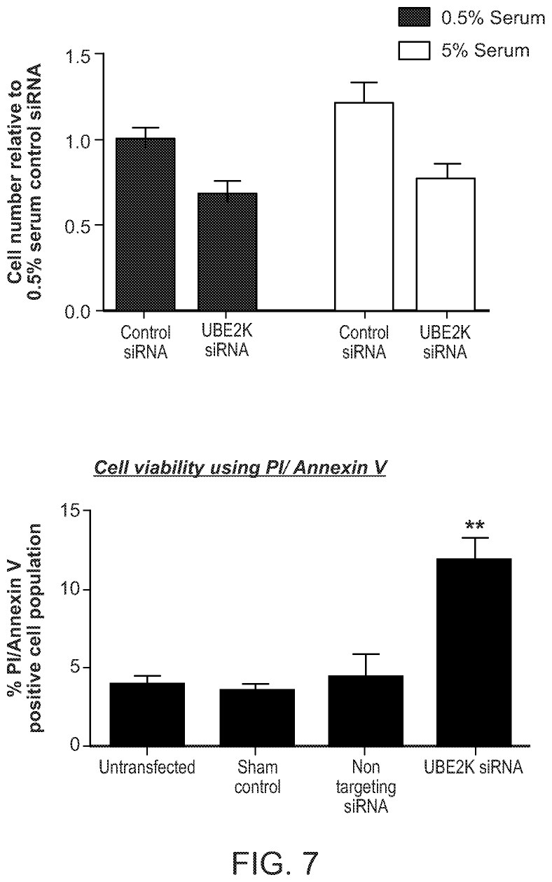

UBE2K knock down in cells may trigger compensatory regulation of other related E2s. To understand this possibility, expression of related E2s was studied upon siRNA-mediated knockdown of UBE2K. Related E2s were selected based on either presence of common E3s for ubiquitination of the substrates (E2D1, E2D2, E2D3) or ability to synthesize Lys 48-linked ubiquitin chains (E2N, cdc34, E2D). E2R2 was also included in the study, as it is closely related to cdc34. Data from Miapaca2 cells are shown in FIG. 6. UBE2K was found to be knocked down at 24 h and 96 h post transfection, and expression of related E2s appeared to be largely unaltered by UBE2K knock down at both time points, suggesting the lack of a compensatory transcriptional response Similar results were obtained in all other cell lines assessed (SKHEP1, HepG2, Hep3B, MDAMB231, BT549, SKBR3, and T47D). Thus siRNA-mediated knockdown of UBE2K does not result in compensatory transcriptional responses of related E2s.

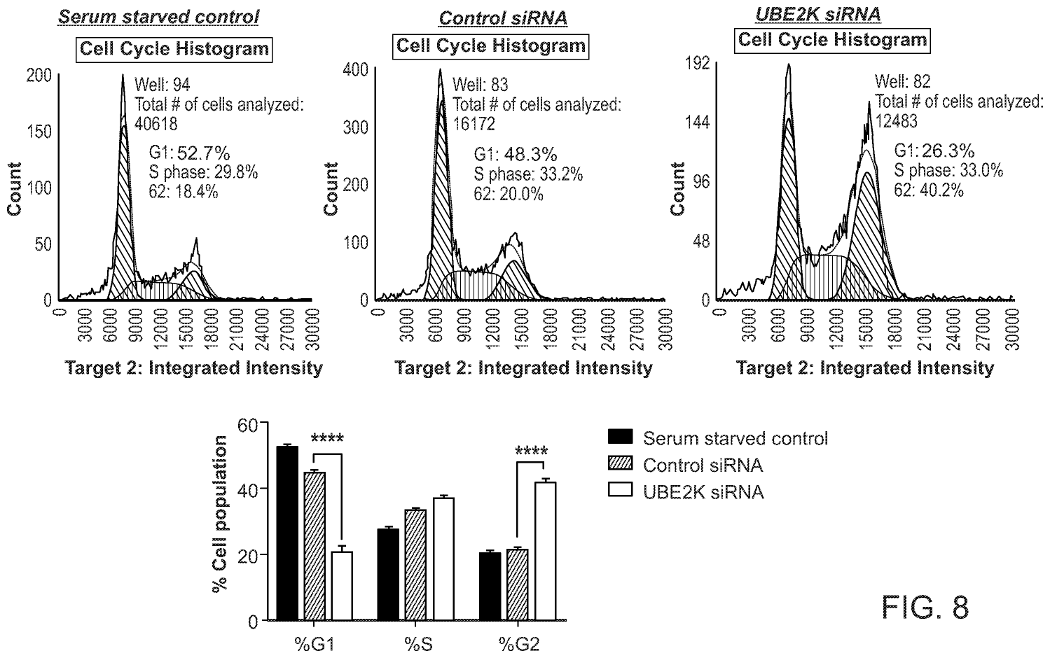

UBE2K is a part of ubiquitin proteasome system, which is responsible for turnover of approximately 80% of proteins in the cells. To investigate if the effect of UBE2K knock down is dependent on the proliferative state of cells, UBE2K knock down Miapaca2 cells were grown in the presence of 5% serum (culture media) or 0.5% serum, and cell number was assessed at 96 h post transfection. As expected, cell number was increased in the 5% serum condition compared to 0.5% serum, indicating serum stimulates proliferation. Nonetheless, UBE2K knock down decreased cell number in both serum conditions to a similar extent (64.1% in 0.5% serum, 64.3% in 5% serum; FIG. 8), indicating the effect of UBE2K knock down is independent of proliferative state.

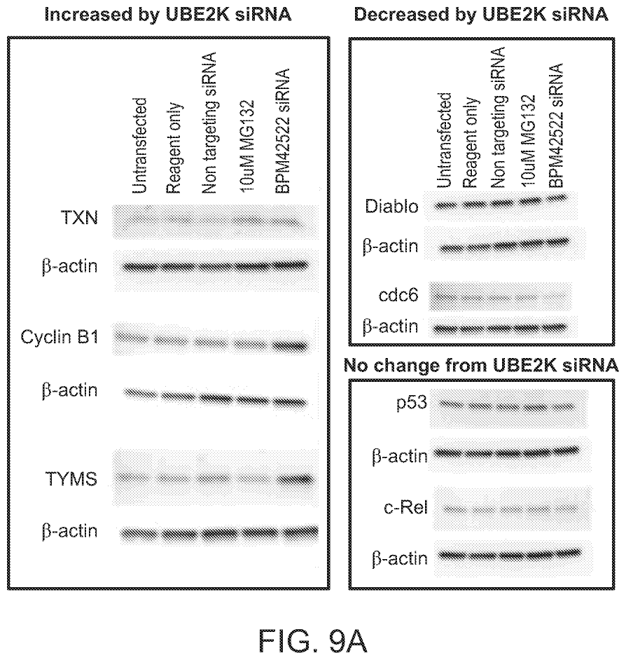

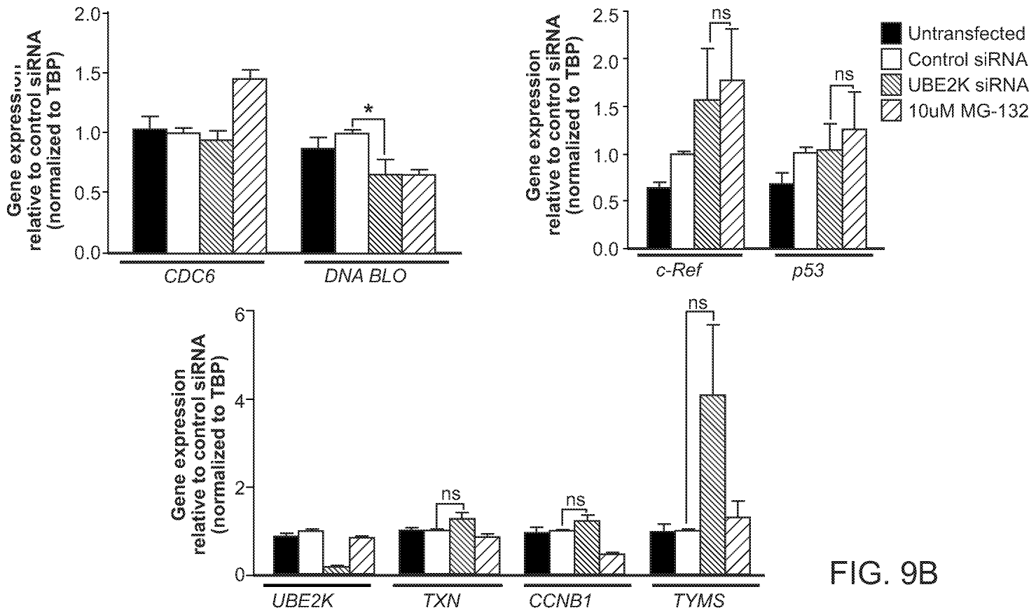

UBE2K knock down in Miapaca2 cells resulted in a 50% decrease in cell number. To understand if this change in cell number is due to increased cell death or decreased cell proliferation, UBE2K siRNA treated versus non targeting siRNA treated cells were assessed using PI/Annexin V and PI cell cycle analysis, respectively. PI/Annexin V data indicated that UBE2K knock down resulted in 6-8% cell death at 96 h post siRNA transfection. Assessment of cell proliferation in UBE2K knock down cells (48 h post transfection) using PI cell cycle analysis revealed that 20% more cells were found in G2/M phase in UBE2K siRNA transfected cells versus non targeting siRNA transfected cells (FIG. 9). This increase in G2/M phase cell population corresponded with a decrease in the population of cells in G1 phase, suggesting that the cells are arrested in G2/M phase. Serum starvation was used as a positive control for cell cycle alterations, resulting in a G1 arrest as expected. Thus, siRNA-mediated knockdown of UBE2K caused a decrease in cancer cell number, the result of a robust G2/M cell cycle arrest, and a modest increase in apoptosis/necrosis.

In order to identify possible UBE2K-dependent substrates which are mechanistically linked to the observed phenotype, a curated list of UBE2K interactors was generated using literature available for UBE2K yeast two hybrid screen/co-immunoprecipitation (Source: Bio Grid). The substrates were selected based on their interactions with other E2s and their role in cell proliferation/cell death (Table 4). Protein levels of 3 out of 7 UBE2K interactors were found to increase in response to UBE2K knock down, two decreased, and two were unchanged (FIGS. 9A and 9B). Expression of these interactors was not found to be altered at the transcript level, suggesting regulation may occur post translationally.

TABLE 4
Curated list of UBE2K interactors

| | | | Interaction | | | |
|---|---|---|---|---|---|---|
| | | | with | Known E3 | | |
| | | E2K | other E2s | interaction | | # of |
| | | as | besides | in UBE2K | | literature |
| Interactor | Function | soleE2 | UBE2K | network | E3 | Evidence |
| Diablo | Inhibitor | Yes | — | MDM2, | — | 2 |
| | of | | | BIRC7and8 | | |
| | apoptosis | | | | | |
| cdc6 | Cell cycle | Yes | — | AnapC11, | — | 2 |
| REL | Oncogene, | No | E2Z | No | — | 1 |
| | apoptosis, | | | | | |
| | immune | | | | | |
| | response | | | | | |
| TP53 | Tumor | No | E2A,B,D1, | MDM2, | — | 2 |
| | suppressor | | I,N,M,J1 | ITCH, | | |
| | | | | BRCA1 | | |
| | | | | UHRF2, | | |
| | | | | RNF2, | | |
| | | | | RING1 | | |
| TXN | Redox | No | 2D2, 2V1 | SIAH1 | — | 1 |
| | cycle | | | | | |
| Cyclin B1 | Cell cycle | No | 2D2 | BRCA1, | — | 1 |
| | | | | UHRF2, | | |
| | | | | ANAPC11 | | |
| TYMS | Target for | No | E2A,E2B, | — | — | 1 |
| | cancer | | E2M,E2C | | | |
| | chemo- | | | | | |
| | therapeutic | | | | | |
| | agents | | | | | |

### Example 3

### siRNA-Mediated Knockdown of UBE2K in Synchronized MiaPaca2 Pancreatic Cancer Cells

The accumulation of cyclin B1 in cells with siRNA-mediated knockdown of UBE2K is consistent with G2/M arrest, as cyclin B1 degradation is required to exit M phase; thus, its levels remain elevated when G2/M arrest occurs. To further discern the mechanistic underpinnings of the effects of UBE2K on cell cycle regulation and, in specific, its role in cyclin B1 degradation, we followed the levels of various cell cycle regulatory proteins over time in synchronized MiaPaca2 cells with and without modulating the expression levels of UBE2K. The experimental setup was as follows; (A) synchronization of MiaPaCa2 cells at G0/G1 phase by serum deprivation and subsequent transfection with siRNA for 48 h (B) releasing the synchronized cells from serum deprivation by adding 2×FBS (20% in DMEM) media for 4 days (C) measurement of cell viability and the levels of various cell cycle regulators including Cyclins D1, El, A2 and B1 and Cdc20.

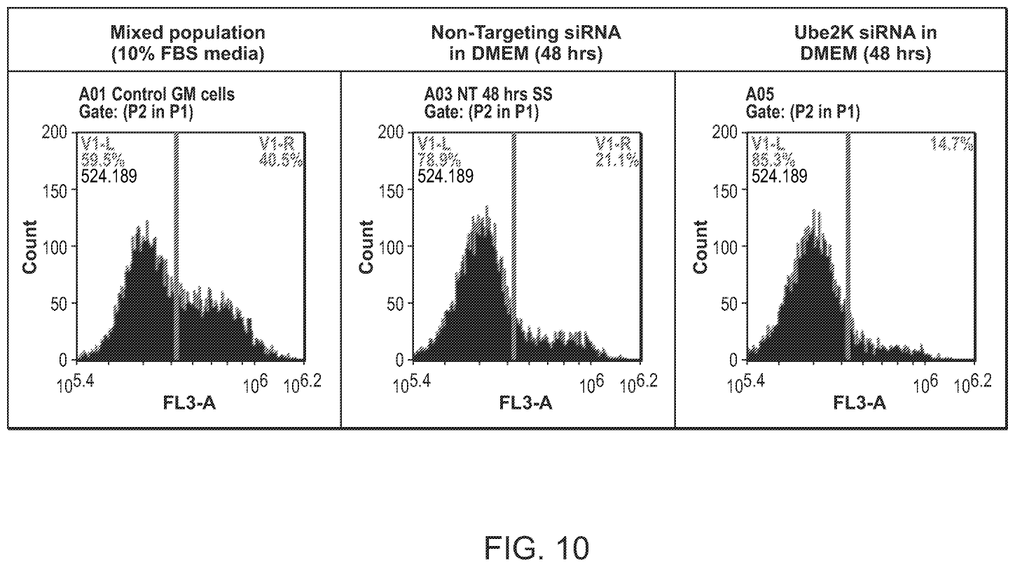

A cell cycle analysis comparing asynchronous population of MiaPaca2 cells (grown in DMEM+10%FBS) with cells that were serum starved for 48 h, revealed that ˜80-85% cells were synchronized at G1 phase due to serum starvation (FIG. 10).

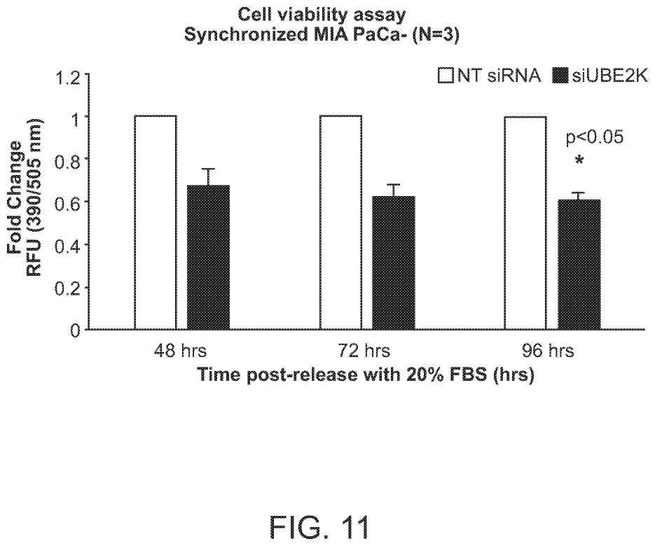

Similar to our observations in unsynchronized MiaPaca2 cell populations, a decrease in the number of viable cells in the siRNA mediated UBE2K knockdown conditions was similarly detected in a synchronized MiaPaCa2 population. Specifically, MiaPaCa-2 UBE2K knockdown cells had ˜40% fewer viable cells than the non-targeting (NT) siRNA controls at 96 hours post-release from synchronization (FIG. 11).

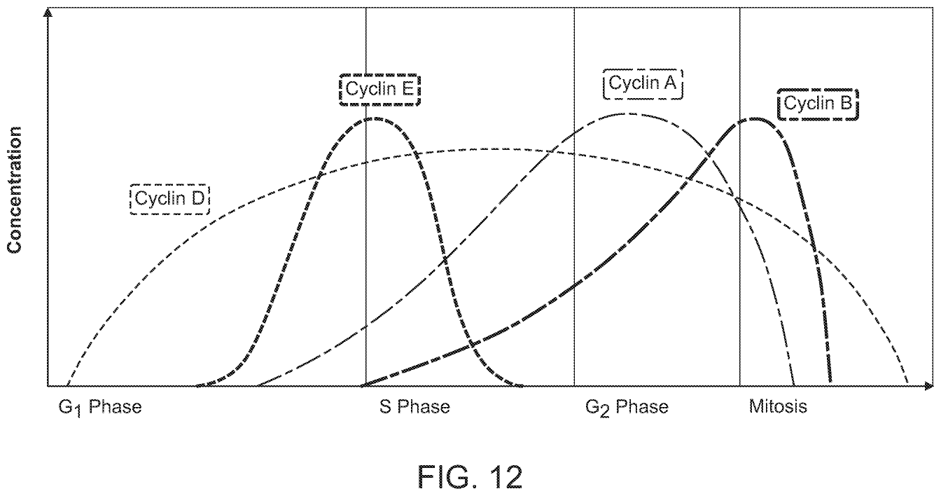

Cyclin family proteins control the progression of cells through the cell cycle by activating cyclin-dependent kinases (Cdks). As the cells progress through different phases of the cell cycle, the cyclin levels decline sharply following each checkpoint (G1/S and G2/M), as cyclins are degraded by cytoplasmic enzymes. A diagram representing the expression levels of human cyclins through different phases of cell cycle is shown in FIG. 12. Cyclin B1, which has been shown to interact with UBE2K, is a regulatory protein involved in mitosis. While cyclin B1 accumulates throughout the cell cycle process and is activated during mitosis, its degradation at the end of mitosis is important for the cell cycle to progress. Cdc20 is a highly conserved activator of the Anaphase Promoting Complex/Cyclosome (APC/C), promoting cell-cycle regulated ubiquitination and proteolysis of a number of critical cell-cycle regulatory targets including the mitotic cyclins (A and B) and Securin.

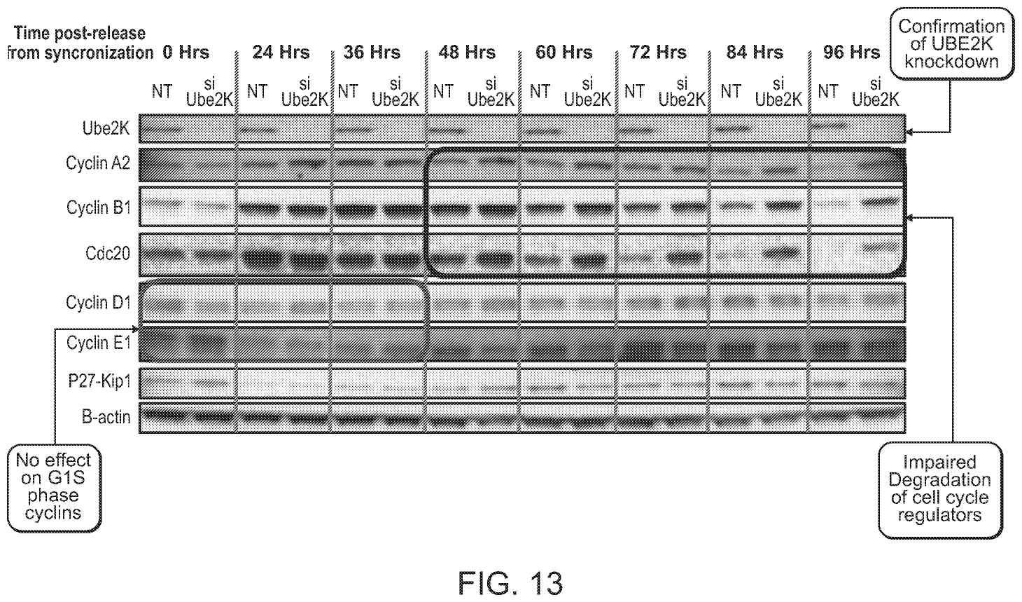

The samples obtained from the cell-synchronization and release were used to perform immunoblotting to check the levels of various cell cycle regulators. Results from the western blots confirmed knockdown of UBE2K with siRNA transfection after 48 hrs. Cyclin E1 levels at the 0 h time point showed that the cells were halted in the G1 phase. Cyclin D1 and E1 levels at 0, 24 and 36 h time points demonstrated cells cycled through the G1 and S phases unperturbed by the knockdown of UBE2K. In contrast, while cyclin A2, cyclin B1 and Cdc20 protein levels accumulated to a similar extent, their levels returned to baseline more slowly in UBE2K siRNA-treated cells, indicating impaired degradation. These observations along with the decrease in the number of viable cells post-release indicates that the loss of UBE2K in MiaPaca2 cells interferes with the Metaphase-Anaphase transition leading to a G2/M arrest in the cells (FIG. 13).

### Example 4

### Comparison of the Effects of UBE2K Knockdown and Cdc34 Knockdown on Viability of MiaPaCa2 Pancreatic Cancer Cells

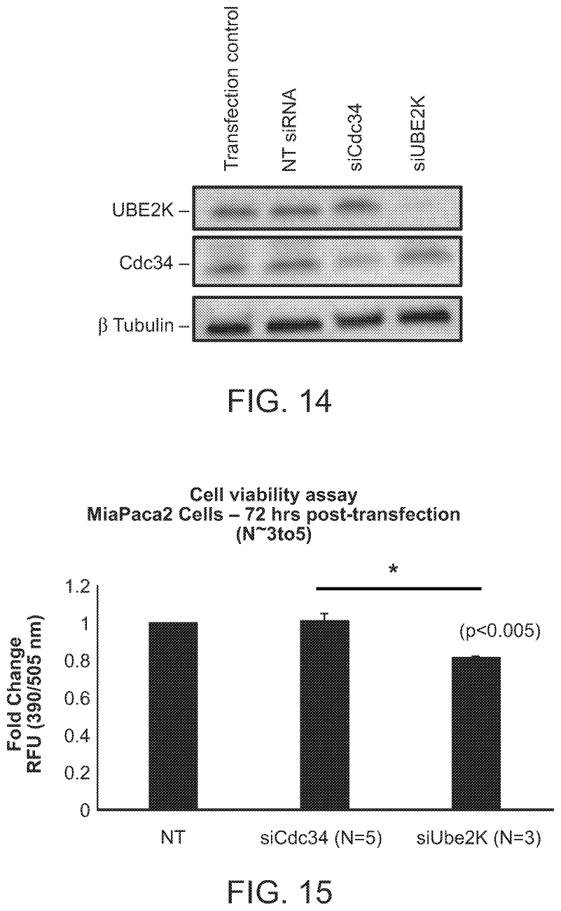

UBE2K is known to preferentially catalyze the formation of Lys48-linked polyubiquitin chains on its substrates leading to their proteasomal degradation. Cdc34 (Ube2R1) is another Lys-48 chain building E2 enzyme in the ubiquitin proteasome system that catalyzes the ubiquitin-mediated degradation of cell cycle G1 regulators and regulates tumor suppressors in multiple cancer types. The results described above show that knocking down UBE2K using targeted siRNA in MiaPaca2 cells resulted in a decrease in the number of viable cells as a result of a robust G2/M cell cycle arrest. Since UBE2K is one among the approximately 50 E2s found in the ubiquitin system, it is important to assess UBE2K specificity in cells. To determine if the above phenotype was specific to the function of UBE2K, the functionally similar E2 enzyme cdc34 was transiently knocked down in MiaPaca2 cells and compared to the effect of UBE2K knockdown. Knockdown was confirmed at the protein level (FIG. 14). As expected, UBE2K knockdown led to a ˜20% decrease in cell viability at 72 hours post-transfection when compared to the NT siRNA treated MiaPaca2 cells. In contrast, Cdc34 knockdown had a minimal effect on the viability of MiaPaca2 cells (FIG. 15), supporting a unique role for UBE2K in this cell type.

### Example 5

### Stable sh-RNA Mediated Knockdown of UBE2K in MiaPaca2 Pancreatic Cells

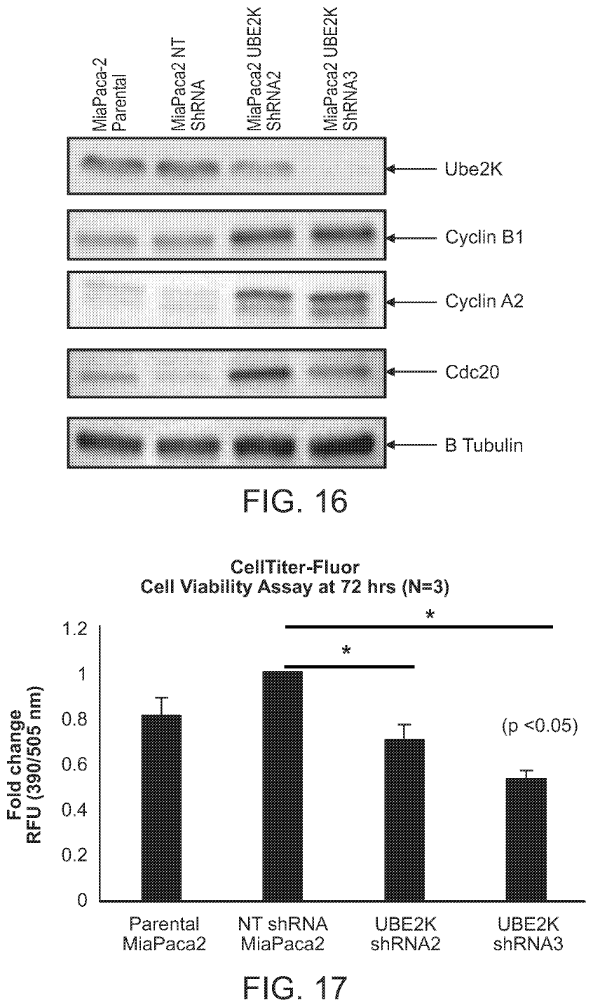

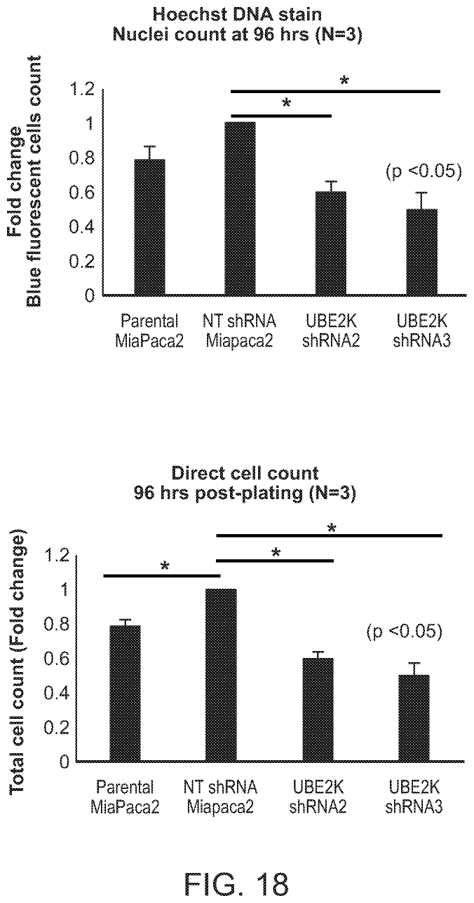

For in vivo proof of concept studies, MiaPaca2 cell lines with stable UBE2K knock down were generated. To this end, Miapaca2 cells were transduced with lentivirus containing shRNA targeted for UBE2K or a non-targeting control. The cell lines were generated as a mixed population of the cells (‘pool of clones’). The efficient knock down of UBE2K was validated at the mRNA (data not shown) and protein level (FIG. 16). Additionally, stable knock down of UBE2K (shRNA 2 and shRNA 3) resulted in increase in protein levels of cell cycle regulators cyclin A2, cyclin B1 and cdc20 (FIG. 16). UBE2K shRNA also induced a slower growth phenotype as depicted by fewer cells or nuclei count in UBE2K stable knock down cells vs non-targeting shRNA transduced cells (FIGS. 17 and 18) 72 h after seeding. Taken together, these data demonstrate key findings observed using siRNA-mediated knockdown of UBE2K are recapitulated in UBE2K stable knock down cells.

### Example 6

### Expression of UBE2K in Human Pancreatic Tumors

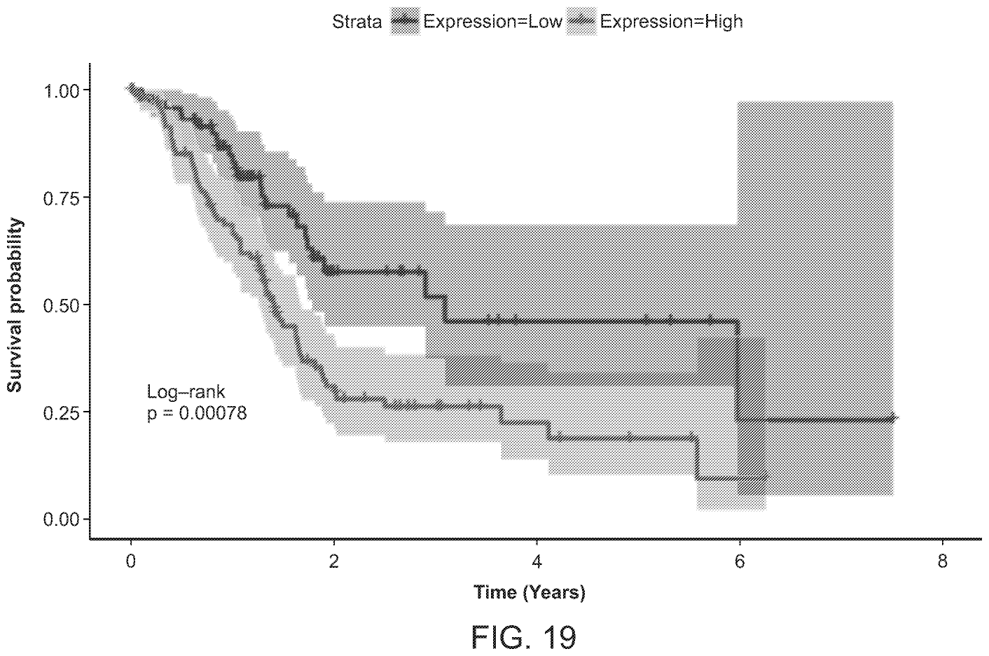

Assessment of UBE2K expression in human pancreatic tumors was performed using two data sets, one from an external publicly-available data set and the other from a commercially available tumor tissue array process. Tumor expression (mRNA) of UBE2K and survival data from patients with pancreatic ductal adenocarcinoma were obtained from the publicly available TCGA database. Results indicated a correlation between lower UBE2K expression and an increase in patient survival (FIG. 19).

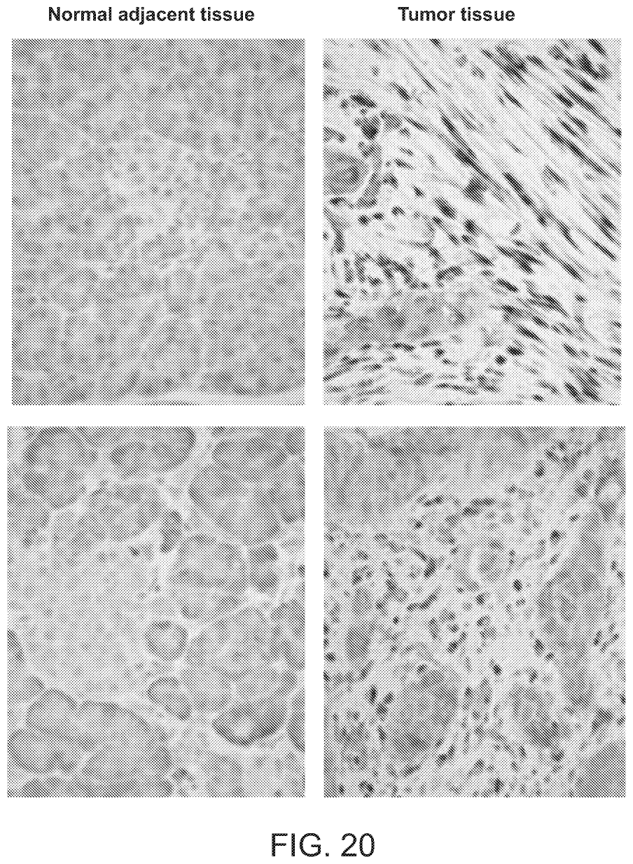

Immunohistochemical analysis of UBE2K expression (protein) using a tissue microarray containing 75 pancreatic tissue samples and matched normal adjacent tissue was performed (FIG. 20). Stained tissues were scored by pathologist and estimated as strong, medium and weak staining. Localization of staining was also scored as membrane, cytoplasmic, or nuclear. Statistical analysis of the staining profiles demonstrated that UBE2K staining was significantly stronger in tumor tissues vs normal adjacent tissues (Table 4, p value <2e-16). Moreover, UBE2K staining to the membrane was observed only in tumor tissues and not in normal adjacent tissues (Table 5, p value <2e-16).

TABLE 5
Statistical analysis of UBE2K staining in normal vs
tumor tissue with regards to subcellular localization

| | TYPE | | | Test | |
|---|---|---|---|---|---|
| | NAT | Malignant | Test | value | p-value |
| STAINING | | | Mann- | 16010 | <2e−16 |
| | | | Whitney test | | |
| None | 6 | 1 | | | |
| Light | 23 | 14 | | | |
| Medium | 27 | 37 | | | |
| Strong | 11 | 15 | | | |
| NUCLEAR | | | Fisher's | | |
| | | | Exact test | | |
| Negative | 67 | 67 | | | |
| Positive | | | | | |
| CYTOPLASMIC | | | Fisher's | | 0.115 |
| | | | Exact test | | |
| Negative | 6 | 1 | | | |
| Positive | 61 | 66 | | | |
| MEMBRANE | | | Fisher's | | <2e−16 |
| | | | Exact test | | |
| Negative | 67 | 18 | | | |
| Positive | | 49 | | | |

### Example 7

### Effects of Small Molecule Inhibitors of UBE2K in Cell Cultures of Human Cancers

Using the methods provided in Example 2 above, the effects of small molecule inhibitors of UBE2K on cell proliferation and cell death of the following human cancer cell lines are determined: choriocarcinoma (JAR), ovarian cancer (PA-1), T cell leukemia (Jurkat clone E6.1), lymphoma (SR), embryonic carcinoma (NCCIT), DB, B cell lymphoma (SU-DHL-6), osteosarcoma (MG63), skin carcinoma (DU4475), T lymphoblast leukemia (MOLT-4), chronic myelogenous leukemia (K562 and KU812), anaplastic large cell lymphoma (SU-DHL-1), and colorectal adenocarcinoma (SW48). It is expected that the small molecule inhibitors of UBE2K will decrease cell proliferation and induce cell death in these human cancer cell lines.

### Example 8

### Effects of Small Molecule Inhibitors of UBE2K in Mouse Models of Human Cancers

Mouse models of the cancers decribed in Example 7 are evaluated to determine the effect of small molecule UBE2K inhibitors on tumor development in vivo. For each mouse model, the human cancer cells (1×10⁷) are suspended in MATRIGEL® and injected into immunocompromised mice. The cancers are allowed to develop for, on average, at least 3 weeks prior to initiation of treatment. For the cancers that form tumors, treatment is not initiated unless palpable tumors were present. The mice are randomized into 2 groups as follows:
i. Group 1—No treatment.
ii. Group 2—Treatment with a small molecule inhibitor of UBE2K
   Mice are observed for viability and secondary symptoms, and tumor growth is monitored by palpation for cancers that form tumors. At mortality, tumors are harvested from the mice, and measured, weighed, and analyzed for the presence of tumor vasculature. It is expected that the small molecule inhibitors of UBE2K will increase survivability of the mice, reduce secondary symptoms, reduce tumor size.

1. A method of treating cancer in a subject in need thereof, the method comprising administering to the subject a Ubiquitin Conjugating Enzyme E2 K (UBE2K) inhibitor, thereby treating the cancer in the subject.

2. A method of reducing proliferation of a cancer cell in a subject in need thereof, the method comprising administering to the subject a Ubiquitin Conjugating Enzyme E2 K (UBE2K) inhibitor, thereby reducing proliferation of the cancer cell in the subject relative to a subject that is not administered the UBE2K inhibitor.

3. A method of inducing death of a cancer cell in a subject in need thereof, the method comprising administering to the subject a Ubiquitin Conjugating Enzyme E2 K (UBE2K) inhibitor, thereby inducing death of the cancer cell in the subject.

4. The method of claim 3, wherein the death of the cancer cell is induced by apoptosis.

5. The method of any one of claims 1-4, wherein the UBE2K inhibitor is a specific inhibitor of UBE2K.

6. The method of any one of claims 1-5, wherein the UBE2K inhibitor comprises a small molecule.

7. The method of any one of claims 1-5, wherein the UBE2K inhibitor comprises a nucleic acid inhibitor.

8. The method of claim 7, wherein the nucleic acid inhibitor comprises an antisense nucleic acid molecule.

9. The method of claim 7, wherein the nucleic acid inhibitor comprises a double stranded nucleic acid molecule.

10. The method of claim 9, wherein the double stranded nucleic acid molecule comprises a double stranded RNA selected from the group consisting of an siRNA, a shRNA, and a dicer substrate siRNA (DsiRNA).

11. The method of any one of claims 1-5, wherein the UBE2K inhibitor comprises an antibody.

12. The method of any one of claims 1-11, wherein the cancer comprises a solid tumor.

13. The method of claim 12, wherein the solid tumor is selected from the group consisting of carcinoma, melanoma, sarcoma, and lymphoma.

14. The method of claim 12, wherein the solid tumor is selected from the group consisting of pancreatic cancer, liver cancer, colorectal cancer, and lymphoma.

15. The method of claim 12, wherein the solid tumor is pancreatic cancer or liver cancer.

16. The method of any one of claims 1-11, wherein the cancer is a leukemia.

17. The method of any of claims 1-16, wherein the UBE2K inhibitor is administered with an additional agent.

18. The method of claim 17, wherein the additional agent is an anti-cancer agent.

19. The method of claim 17, wherein the additional agent is a chemotherapeutic agent.

20. The method of claim 19, wherein the chemotherapeutic agent is selected from the group consisting of gemcitabine, 5-fluorouracil, leucovorin, docetaxel, fludarabine, cytarabine, cyclophosphamide, paclitaxel, docetaxel, busulfan, methotrexate, daunorubicin, doxorubicin, melphalan, cladribine, vincristine, vinblastine, chlorambucil, tamoxifen, taxol, camptothecin, actinomycin-D, mitomycin C, combretastatin, cisplatin, etoposide, verapamil, podophyllotoxin, and 5-fluorouracil.

21. The method of claim 17, wherein the additional agent is an anti-angiogenic agent.

22. The method of claim 17, wherein the additional agent is an immunotherapeutic.

23. The method of claim 22, wherein the immunotherapeutic is an immune checkpoint modulator of an immune checkpoint molecule.

24. The method of 23, wherein the immune checkpoint molecule is selected from CD27, CD28, CD40, CD122, OX40, GITR, ICOS, 4-1BB, ADORA2A, B7-H3, B7-H4, BTLA, CTLA-4, IDO, KIR, LAG-3, PD-1, PD-L1, PD-L2, TIM-3, and VISTA.

25. The method of claim 23, wherein the immune checkpoint molecule is a stimulatory immune checkpoint molecule and the immune checkpoint modulator is an agonist of the stimulatory immune checkpoint molecule.

26. The method of claim 23, wherein the immune checkpoint molecule is an inhibitory immune checkpoint molecule and the immune checkpoint modulator is an antagonist of the inhibitory immune checkpoint molecule.

27. The method of any one of claims 23-26, wherein the immune checkpoint modulator is selected from a small molecule, an inhibitory RNA, an antisense molecule, and an immune checkpoint molecule binding protein.

28. The method of claim 23, wherein the immune checkpoint molecule is PD-1 and the immune checkpoint modulator is a PD-1 inhibitor.

29. The method of claim 28, wherein the PD-1 inhibitor is selected from pembrolizumab, nivolumab, pidilizumab, SHR-1210, MEDI0680R01, BBg-A317, TSR-042, REGN2810 and PF-06801591.

30. The method of claim 23, wherein the immune checkpoint molecule is PD-L1 and the immune checkpoint modulator is a PD-L1 inhibitor.

31. The method of claim 30, wherein the PD-L1 inhibitor is selected from durvalumab, atezolizumab, avelumab, MDX-1105, AMP-224 and LY3300054.

32. The method of claim 23, wherein the immune checkpoint molecule is CTLA-4 and the immune checkpoint modulator is a CTLA-4 inhibitor.

33. The method of claim 32, wherein the CTLA-4 inhibitor is selected from ipilimumab, tremelimumab, JMW-3B3 and AGEN1884.


SEQUENCE LISTING (2 sequences)

SEQ ID NO: 1 - protein, 200 aa, Homo sapiens
MANIAVQRIKREFKEVLKSEETSKNQIKVDLVDENFTELRGEIAGPPDTPYEGGRYQLEIKIPETYPFNPPKVRFITKIWHPNISSVTGAICLDILKDQWAAAMTLRTVLLSLQALLAAAEPDDPQDAVVANQYKQNPEMFKQTARLWAHVYAGAPVSSPEYTKKIENLCAMGFDRNAVIVALSSKSWDVETATELLLSN

SEQ ID NO: 2 - DNA, 603 bp, Homo sapiens
atggccaacatcgcggtgcagcgaatcaagcgggagttcaaggaggtgctgaagagcgaggagacgagcaaaaatcaaattaaagtagatcttgtagatgagaattttacagaattaagaggagaaatagcaggacctccagacacaccatatgaaggaggaagataccaactagagataaaaataccagaaacatacccatttaatccccctaaggtccggtttatcactaaaatatggcatcctaatattagttccgtcacaggggctatttgtttggatatcctgaaagatcaatgggcagctgcaatgactctccgcacggtattattgtcattgcaagcactattggcagctgcagagccagatgatccacaggatgctgtagtagcaaatcagtacaaacaaaatcccgaaatgttcaaacagacagctcgactttgggcacatgtgtatgctggagcaccagtttctagtccagaatacaccaaaaaaatagaaaacctatgtgctatgggctttgataggaatgcagtaatagtggccttgtcttcaaaatcatgggatgtagagactgcaacagaattgcttctgagtaactga